(19) United States
(12) Patent Application Publication
(10) Pub. No.: US 2002/0150966 A1
(43) Pub. Date: Oct. 17, 2002

(54) Specimen-linked database

(76) Inventor: Patrick J. Muraca, Pittsfield, MA (US)

Correspondence Address: PALMER & DODGE, LLP, PAULA CAMPBELL EVANS, 111 HUNTINGTON AVENUE, BOSTON, MA, 02199, US

(21) Appl. No.: 09/781,016

(22) Filed: Feb. 9, 2001

(51) Int. Cl. G06F 19/00; G01N 33/48; G01N 33/50; G01N 1/30

(52) U.S. Cl. 435/40.5; 702/19

(57) ABSTRACT

A user of a tissue microarray is provided with access to a specimen-linked database comprising patient and tissue information for the samples located on the microarray. In one embodiment, access to the database is obtained through a tissue information system comprising at least one device which is connectable to the network. The tissue information system enables the user to search and identify relationships between molecular profiling information obtained for the microarray and patient information, allowing the user to obtain diagnostic and prognostic information, to identify drug targets, and to validate drug leads which interact with these targets. The invention further provides a system for ordering customized tissue microarrays.

### FIELD OF THE INVENTION

The invention relates to a method and system for accessing, organizing, and displaying tissue information. In particular, the invention relates to a method and system for correlating molecular profiling data obtained from tissue microarrays with patient information in a specimen-linked database. In one embodiment, the tissue microarrays comprise tissue samples obtained from autopsy samples and the tissue information includes cause of death.

### BACKGROUND OF THE INVENTION

The ability to monitor disease progression is an important tool in medicine because it allows a physician to select the most appropriate course of treatment for a particular disease or combination of diseases. The responsiveness of a disease to a particular therapy can be affected by such factors as drug selection and dosage, the genetic makeup, age, and sex of the patient, as well as demographic, and/or environmental factors. These factors may also contribute to the side effects of a particular drug therapy. Often, the role of less quantifiable variables, such as the lifestyle or environment of the patient, can't be appreciated until connections can be identified between these variables and a disease state and/or with molecular profiling data used to characterize a disease state. It is desirable to have as much information as possible at the beginning of medical treatment, because providing more details enables a physician to identify specific disease states with greater accuracy.

In practice, the information obtained by a physician prior to drug selection has generally been limited to obtaining the patient's medical history. Medical history can be unreliable, as it is usually obtained just prior to beginning treatment, when the patient may be under stress, or may not be able to provide all of the available information needed by the physician. Molecular profiling data from tissue samples obtained the patient (e.g., biopsies) can greatly expand a physician's knowledge base because this data can be correlated with molecular profiling data and clinical information from other patients (e.g., data from other living patients or from autopsy information). The sequencing of the human genome has provided thousands of molecular probes useful for generating molecular profiling data. However, while there is no shortage of molecular and clinical information that can be obtained from tissue samples from living patients or autopsy tissue samples, the development of systems and methods for managing this information to determine its biological relevance (i.e., to identify meaningful diagnostic correlations) has lagged behind.

Genomic information retrieval databases coupled to database search systems exist. An example is the National Center for Biotechnology Information (NCBI) Database (www.ncbi.nlm.nih.gov/entrez). Upon accessing the NCBI website an interface is displayed which provides links to a number of other databases, e.g., a scientific literature database (PubMed); a nucleotide sequence search and retrieval database (Entrez Nucleotides); a protein sequence search and retrieval system; a genome sequence database (Entrez Genomes); a Molecular Modeling Database (MMDB); a population database (e.g., comprising aligned sequences submitted as a set resulting from a population a phylogenetic, or mutation study describing such events as evolution and population variation); and a taxonomy database, which provides hyperlinks to sources of phylogenetic information. However, the NCBI databases do not provide information about tissue standards, or about patient information, and do not provide a way to correlate molecular profiling data with patient information.

Some tissue banks, such as the American Type Culture Collection (ATCC®), provide both tissue samples and computer accessible information about the tissues they bank. For example, the ATCC database provides a searchable database relating to an extensive cell line collection. The ATCC database is accessible through an interface displayed on the website, www.atcc.org, and comprises a series of links relating to a variety of ATCC products. Selecting a link will display an interface which provides additional links providing more detailed information about a particular product. In one embodiment, links representing different cell lines are displayed. Clicking on one of these links will display information such as the organism from which the particular cell line is derived, the tissue type, and limited patient information (e.g., age, ethnicity, and gender of the individual from whom the cell line was generated). The database and display system do not provide a convenient way to access both tissue information and molecular data relating to a particular tissue source (e.g., a cell line), and do not provide images of morphological features relating to the cells of the particular cell line.

There have also been efforts to create data retrieval databases for autopsy information. The creation of a computerized central database for autopsy information was first attempted by the College of American Pathologists in 1975 in their effort to create the National Autopsy Databank. The effort was frustrated by the lack of adequate computer technology at the time and the lack of availability of computers. An additional problem was the large volume of information that needed to be entered into this database, and the daunting clerical effort required to enter and encode the information. In 1996, Moore, et al., A Prototype Internet Autopsy Database, Arch. Pathol. Lab. Med., 120:728, 1996, proposed the use of an Internet autopsy database, to make autopsy information more accessible to clinicians.

Other databases which catalog medical findings into computer format include the Neuropathology Database of the Boston University Alzheimer Disease Center (McKee et al., Brain Banking: Basic Science Methods, Alzheimer Disease and Associated Disorders, 13:539, 1999). A website posted by The Department of Pathology at the University of Pittsburgh (www.path.upmc.edu) provides an interface displaying links which identify particular cases assessed by the Department of Pathology. Selecting a link displays an interface which provides an image of a tissue sample from a patient and a limited amount of the patient's medical history (e.g., age, gender, symptoms presented) as well as images of tissue biopsies from the same patient stained with a variety of antibodies. This interface comprises an additional link, “Final Diagnosis.” Selection of the “Final Diagnosis” link displays another interface which summarizes the disease diagnosed and features unique to the particular patient samples provided. The database does not provide a way to correlate new data with the existing data within the database, or to identify relationships between biological characteristics of the tissue samples and multiple patients.

### SUMMARY OF THE INVENTION

There is a need in the art for methods and systems for accessing, organizing, and displaying tissue information. The invention provides information about tissues in an interactive format which allows for searching, comparison, relationship determination, organization, and display of information.

In one aspect, the invention provides panels of tissue standards along with access to an tissue information system. In one embodiment according to this aspect, the tissue information system comprises a specimen-linked database which is in communication with an information management system. The specimen-linked database is a repository of information including, but not limited to, information relating to phenotype, genotype, pathology, and expression of biomolecules in tissues, and including information relating to the medical history of the individuals who are the sources of tissues being analyzed. The database also provides demographic and epidemiologic information on populations of individuals who provide tissues which have been, or are being, analyzed.

In one embodiment, the information management system which is coupled to the database includes database search and relationship determination functions. The database search function enables the user to design queries to obtain information about tissues in the database, while the relationship determination function enables the user to identify relationships between different biological characteristics of tissues (e.g., the relationship between the expression of biomolecules and patient information). Relationships so determined can be stored in a relational subdatabase of the database.

In one embodiment, the relationship determination function of the information management system enables the user to link gene sequence information in the database to information about the function of the gene to clinical information about a tissue source expressing the gene. In another embodiment, the user can generate his or her own links and customize the information stored in a personal relational subdatabase portion of the database.

In one embodiment of the present invention, the panels of tissues which are the source of information in the database are organized onto substrates as microarrays. Microarrays according to the invention comprise a plurality of tissue samples, each sample stably associated with a different sublocation on the substrate, and each sample comprising at least one known biological characteristic (e.g., such as tissue type). In one embodiment of the invention, the microarray comprises from 2-1000 sublocations. In another embodiment, the microarray comprises greater than 500 sublocations, or greater than 1000 sublocations. In a further embodiment of the invention, at least 50% of the sublocations comprise different tissue types.

Sources of tissues which form the sublocations of the microarrays include human tissue, non-human tissue (animals and/or plants), diseased tissues, normal tissues, and tissues which comprise mixtures of diseased and normal cells. In some embodiments, the microarray comprises tissues representing the entire body of a single individual; tissues from populations of individuals, tissues representing different developmental stages, and tissues expressing recombinant nucleic acids (e.g., comprising different copy numbers of the same or different genes). In one embodiment, the tissue microarray comprises tissues which represent different stages in the progression of a disease; e.g., the disease is a cell proliferative disorder, such as cancer.

In one embodiment, the tissue microarrays comprise tissues obtained from autopsies, or other surgical procedures in which the patient died. In this embodiment, the microarrays are provided to a user along with access to a database comprising information such as the type of drugs that the patient was taking when he or she died, the cause of death, underlying diseases, medical history, family relationships, as well as any molecular profile data available. In another embodiment, information obtained during subsequent examination of the tissues (e.g., by clinicians throughout the world) is added to the database, providing a dynamic database which reflects large-scale population data.

In another embodiment, a completely random selection of tissues is used to construct the tissue microarray, and the information provided by the database is used to evaluate the results obtained during a screen for common properties of the tissues or common medical information about the tissue sources, enabling the user to correlate a molecular and/or clinical profile with a particular disease state.

The tissue microarrays can be used to obtain diagnostic and/or prognostic information, information relating to disease recurrence, and epidemiological information. In other embodiments, the microarrays are used to evaluate the effects of an environmental condition (e.g., such as an environmental hazard), a therapeutic agent (e.g., a drug), a potentially toxic agent, or even of a pattern of behavior. The microarrays can also be used to identify the biological targets of therapeutic agents and, in conjunction with the database and information management system, can be used to prioritize these targets.

In some embodiments, tissue microarrays are analyzed in conjunction with nucleic acid microarrays, peptide microarrays, and/or other small biomolecule arrays. In one aspect of this embodiment, the nucleic acids, peptides, and small biomolecules are obtained from the same patient (and even tissue type) as the tissue samples in the tissue microarray. In this embodiment, access to the database includes providing access to molecular profiling data obtained from any or all of these arrays, as well as providing access to clinical or demographic information on the patient who is the source of the tissue, nucleic acids, peptides, and/or small biomolecules.

In one embodiment, accessing the database is mediated through a tissue information system which provides at least one user device connectable to the network (e.g., a computer or wireless device) which can communicate with the specimen-linked database and information management system (e.g., through a server and linking program(s)). In one embodiment, the user device comprises an operating system and one or more application programs, including an Internet browser, for accessing the network. In another embodiment, the tissue information system comprises at least one server which comprises data storage media for maintaining the database. The server itself can include one or more applications, including the information management system.

In one embodiment, a user is provided with access to the specimen-linked database by being provided with information as to how to communicate with the information management system. For example, in one embodiment, the user is provided with the address (e.g., a URL) of a web page interface which the user accesses by communicating with the network. In one embodiment, accessing the web page interface enables the user to access the server which includes the information management program.

In another embodiment, providing access to the user further includes providing the user with an identifier which identifies a particular microarray about which the user desires information. When the user communicates the identifier to the tissue information system (e.g., inputting characters representing the identifier into a field displayed on the web page interface), an interface is displayed which provides a plurality of selectable coordinates. Each coordinate represents a tissue at a particular sublocation on the microarray being analyzed and each coordinate is associated with a link for accessing the specimen-linked database. In one embodiment, when the user selects the link corresponding to a particular coordinate, information relating the tissue at a sublocation corresponding to that coordinate is displayed. In another embodiment, when the user selects the link, an interface providing information categories is displayed; each information category description associated with a link to a portion of the database comprising information relating to the information category. Both information and information categories can be displayed on a single interface.

In one embodiment of the invention, the tissue information system provides an interface which presents a representation of the tissue array. In one embodiment, images of tissue samples at each sublocation are provided. In this embodiment, the images themselves may provide a graphical representation of coordinates (i.e., clicking on an image of a sublocation will link the user to the information relating to the tissue at that sublocation). However, in another embodiment, coordinate links are displayed in proximity to the image of the tissue at the sublocation. In a further embodiment; the user is presented with field(s) into which the user inputs the coordinates of particular sublocation(s) the user desires access to information about, and the system displays the information and/or further links to information categories in response to this inputting.

In another embodiment, when the user accesses the database, an interface is displayed which communicates with a diagnostic matrix subdatabase (a relational subdatabase which relates the expression of a gene (e.g., cancer) to a particular disease state (e.g., the stage or grade of cancer)). In this embodiment, the interface enables the user to input information relating to the expression of biological characteristic(s) (e.g., gene expression, protein expression, the expression of morphological characteristic(s), and the like) and to communicate the information to the tissue information system. The information management system then retrieves information from the specimen-linked database about the disease state associated with the particular expression pattern identified by the user. In one embodiment, the information management system provides information relating to diagnosis, prognosis, or likelihood of recurrence of a disease, based upon the correlation of the expression pattern and the disease state.

In one embodiment, the tissue information system displays diagnostic, prognostic, or disease recurrence information. However, in another embodiment, the system provides a report comprising this information to the user. The report may be in a written, electronic, or verbal form. In a further embodiment of the invention, the information displayed, and/or the report provided, includes information relating to clinical trials providing treatment options, information relating to FDA approved treatment options appropriate for a particular disease diagnosis or prognosis; and/or contact information including the names of physicians who may provide additional treatment information.

In one embodiment, the tissue information system comprising the database and information management system is used to prioritize drug targets. In this embodiment, data relating to the expression of biological characteristics by tissues at different sublocations on a microarray (i.e., molecular profiling data) are communicated to the tissue information system, e.g., by inputting the information into a “new information” interface displayed by the system, or through an automated molecular profiling system comprising a processor which automatically provides information to the tissue information system. The information management system then implements its relationship determining function to identify relationships between an individual biological characteristic, or sets of biological characteristics, and a disease. Biological characteristics which are highly related to the disease (e.g., show a statistically significant correlation) are identified as drug targets, and agents which affect the expression of these biological characteristics are screened for to identify drug leads for treating the disease.

In another embodiment, the tissue information system is also used in the drug screening process. In one embodiment, tissue microarray(s) are used to determine the presence and/or location of a drug lead within tissue(s), and the user communicates this information to the tissue information system. In one embodiment, the tissue information system assigns values to the drug leads tested, with a high value being assigned to a drug lead which is expressed only in tissues affected by the disease. In another embodiment, the tissue information system further determines relationships between drug leads and patient data (e.g., toxicity information, information concerning efficacy, adverse effects, half-life of the drug lead in the patient's circulation, and the like), ranking drug leads which have low numbers of adverse effects and/or adverse effects which are not severe, and a long half-life (or a half life having a selected value) with high values, and drug leads which have high adverse effects and/or severe adverse effects, and a short half-life (compared to a selected value) with low values. In this embodiment, the information management system displays identifiers identifying the drug leads, ordering them according to their rank. Selecting particular identifier(s) will cause information relating to particular drug leads to be displayed.

The invention further provides a system for ordering customized microarrays electronically. In one embodiment, a first user is provided access to an interface which displays identifiers, each of which identifies a different tissue type. The first user identifies tissue types of interest (e.g., by checking any of a plurality of boxes provided along side an identifier which identifies the tissue type), or obtains more information about the tissue types (e.g., in this embodiment, the tissue type identifier is itself a link which, when selected, displays information about the tissue type, such as patient data, molecular profile data, and the like). In one embodiment, the interface further provides an option to select tissue type(s) as well as the option to select more links, or to continue searching to identify other tissues of interest. Selection of tissue type(s) is communicated to a microarray generator which constructs the tissue microarray.

In another embodiment, the interface further requests information from the first user such as billing information (credit card, account number, and the like), address, date required, and other shipping information. In further embodiments, the user is also provided with the option to select nucleic acid arrays, peptide arrays, and/or other small biomolecule arrays, which may be arrayed on the same or different substrates as the tissue microarray.

The invention further contemplates embodiments where the invention is provided as a kit. The kit minimally contains a tissue microarray and provides access to an information database (e.g., in the form of a URL and an identifier which identifies the particular microarray being used). In another embodiment, kit comprises instructions for accessing the database, or one or more molecular probes for obtaining molecular profiling data using the microarray, and/or other reagents necessary for performing this analysis (e.g., labels, suitable buffers, and the like). In one embodiment, the components of the kit are customized according to the needs of a user, e.g., assembled by a second user after receiving information from a first user whose has accessed a system according to the invention.

### BRIEF DESCRIPTION OF THE DRAWINGS

The objects and features of the invention can be better understood with reference to the following detailed description and accompanying drawings.

FIG. 1A shows a flow chart according to one embodiment of the invention in which tissue microarrays according to the invention are used in conjunction with gene chips to identify, prioritize, and validate drug targets. FIG. 1B shows a schematic diagram of how data from a microarray is used in this process.

FIG. 2A is an illustration of a profile microarray substrate according to one embodiment of the invention, comprising a first location for placing a tissue sample and a second location comprising a microarray. Each sublocation on the microarray represents a different stage of breast cancer. FIG. 2B shows an microarray locator according to one embodiment of the invention next to a profile microarray substrate, for determining the coordinates of different sublocations on the microarray. FIG. 2C shows six different sublocations from the microarray shown in FIG. 2A. Each sublocation represents different stages of breast cancer stained with a CK7 antibody. FIG. 2D shows a profile microarray substrate comprising a test tissue at a first location and a microarray at a second location. The test tissue is stained with a breast cancer specific antibody. FIG. 2D shows information provided in a kit which comprises the profile microarray substrate shown in FIG. 2A and the microarray locator shown in FIG. 2B.

FIG. 3 shows a tissue microarray according to the present invention comprising a plurality of sublocations, each sublocation comprising a tissue sample whose morphological features can be distinguished under a microscope.

FIGS. 4A-4C show an interface on a display of a user device connectable to a network which displays information relating to the biological characteristics of tissues at different sublocations in a tissue microarray. FIG. 4A shows an interface for addressing a breast cancer microarray and for inputting new information relating to the tissue samples in the microarray into a database. FIG. 4B shows a display of a portion of the database. FIG. 4C shows a display on the interface of the device which displays relationships identified between medical data and molecular profiles obtained for tissue samples on the tissue microarray.

FIG. 5 is a schematic diagram illustrating a system comprising a specimen-linked database and information management system according to one embodiment of the invention.

FIG. 6 is a flow chart showing a method according to one embodiment of the invention, for organizing and displaying tissue information obtained from a tissue microarray.

FIGS. 7A-G show interfaces on the display of a user device connectable to the network for organizing a displaying information relating to tissue microarrays.

FIG. 8 shows an optical system according to one embodiment of the invention for detecting and processing optical information from a tissue microarray.

FIG. 9 shows components of a system used to order customized microarrays according to one embodiment of the invention.

FIG. 10 illustrates an interface on a display of a user device, according to one embodiment, for accessing a genomics medicine database in the system.

FIG. 11 illustrates an interface on a display of a user device, according to one embodiment, displaying relationships identified by the system.

FIG. 12 is a flow chart showing a method of validating information included in the database.

FIG. 13 shows exemplary SNOMED® anatomical code numbers used to cross-reference tissue specimens linked to the database according to one embodiment of the invention.

FIGS. 14A, B and C show exemplary SNOMED® diagnostic codes used to cross-reference information about tissue specimens linked to the database according to one embodiment of the invention.

FIG. 15 shows an exemplary data table obtained using the system of the invention, in which information about tissue specimens is cross-referenced to the database using ICD-9-CM and DSM-IV-TR codes, in one embodiment of the invention.

### DESCRIPTION

The invention relates to a method and system for accessing, organizing, and displaying tissue information obtained from tissue microarrays. The method and system according to the invention enables the user to correlate molecular profiling data with patient information, including, in some embodiments, cause of death. Various or all of the steps of the process, including the steps of obtaining molecular information, can be automated. In one embodiment of the invention, the user is provided with access to a specimen-linked database allowing him or her to customize a tissue microarray and order that microarray online.

### Definitions

In order to more clearly and concisely describe and point out the subject matter of the claimed invention, the following definitions are provided for specific terms which are used in the following written description and the appended claims.

As used herein, the term “information about the patient” refers to any information known about the individual (a human or non-human animal) from whom a tissue sample was obtained. The term “patient” does not necessarily imply that the individual has ever been hospitalized or received medical treatment prior to obtaining a tissue sample. The term “patient information” includes, but is not limited to, age, sex, weight, height, ethnic background, occupation, environment, family medical background, the patient's own medical history (e.g., information pertaining to prior diseases, diagnostic and prognostic test results, drug exposure or exposure to other therapeutic agents, responses to drug exposure or exposure to other therapeutic agents, results of treatment regimens, their success, or failure, history of alcoholism, drug or tobacco use, cause of death, and the like). The term “patient information” refers to information about a single individual; information from multiple patients provides “demographic information,” defined as statistical information relating to populations of patients, organized by geographic area or other selection criteria, and/or “epidemiological information,” defined as information relating to the incidence of disease in populations.

As defined herein, the term “information relating to” is information which summarizes, reports, provides an account of, and/or communicates particular facts, and in some embodiments, includes information as to how facts were obtained and/or analyzed.

As used herein, the term, “in communication with” refers to the ability of a system or component of a system to receive input data from another system or component of a system and to provide an output in response to the input data. “Output” may be in the form of data or may be in the form of an action taken by the system or component of the system.

As used herein, the term “provide” means to furnish, supply, or to make available.

As defined herein, “an individual” is a single organism and includes humans, animals, plants, multicellular and unicellular organisms.

As defined herein, “an identical tissue type” is one which shares the same developmental origins as another tissue type.

As defined herein, a “tissue” is an aggregate of cells that perform a particular function in an organism. The term “tissue” as used herein refers to cellular material from a particular physiological region. The cells in a particular tissue may comprise several different cell types. A non-limiting example of this would be brain tissue that further comprises neurons and glial cells, as well as capillary endothelial cells and blood cells. The term “tissue” also is intended to encompass a plurality of cells contained in a sublocation on the tissue microarray that may normally exist as independent or non-adherent cells in the organism, for example immune cells, or blood cells. The term is further intended to encompass cell lines and other sources of cellular material that now exist which represent specific tissue types (e.g., by virtue of expression of biomolecules characteristic of specific tissue types).

As defined herein, a “molecular probe” is any detectable molecule, or is a molecule which produces a detectable molecule upon reacting with a biological molecule. “Reacting” encompasses binding, labeling, or catalyzing an enzymatic reaction. A “biological molecule” is any molecule which is found in a cell or within the body of an organism.

As used herein, the term “biological characteristics of a tissue” refers to the phenotype and genotype of the tissue or cells within a tissue, and includes tissue type, morphological features; the expression of biological molecules within the tissue (e.g., such as the expression and accumulation of RNA sequences, the expression and accumulation of proteins (including the expression of their modified, cleaved, or processed forms, and further including the expression and accumulation of enzymes, their substrates, products, and intermediates); and the expression and accumulation of metabolites, carbohydrates, lipids, and the like). A biological characteristic can also be the ability of a tissue to bind, incorporate, or respond to a drug or agent. “Biological characteristics of a tissue source” are the characteristics of the organism which is the source of the tissue (e.g., such as the age, sex, and physiological state of the organism).

As defined herein, “a diagnostic trait” is an identifying characteristic, or set of characteristics which in totality are diagnostic. The term “trait” encompasses both biological characteristics and experiences (e.g., exposure to a drug, occupation, place of residence). In one embodiment, a trait is a marker for a particular cell type, such as a transformed, immortalized, pre-cancerous, or cancerous cell, or a state (e.g., a disease) and detection of the trait provides a reliable indicia that the sample comprises that cell type or state. Screening for an agent affecting a trait thus refers to identifying an agent which can cause a detectable change or response in that trait which is statistically significant.

As defined herein, a “reliable indicia” refers to an indicia which is both specific and sensitive in its ability to diagnose a cell type or state. In one embodiment, an indicia is reliable if it is capable of detecting positive occurrences of a cell type or state greater than 70% of the time, and falsely identifies occurrences of a cell type or state less than 20% of the time. In a preferred embodiment, a reliable indicia is one which detects positive occurrences of a cell type or state greater than 90% of the time and falsely identifies occurrences of a cell type or state less than 5% of the time.

A “disease or pathology” is a change in one or more biological characteristics that impairs normal functioning of a cell, tissue, and/or organism.

As defined herein, “a cell proliferative disorder” is a condition marked by any abnormal or aberrant increase in the number of cells of a given type or in a given tissue. Cancer is often thought of as the prototypical cell proliferative disorder, yet disorders such as atherosclerosis, restenosis, psoriasis, inflammatory disorders, some autoimmune disorders (e.g., rheumatoid arthritis) are also caused by abnormal proliferation of cells, and are thus also examples of cell proliferative disorders.

As used herein, the term “course of disease” refers to the sequence of events in which a disease develops, causes symptoms, and is either recovered from, or continues, and/or increases in severity.

As used herein, the term “cancer” refers to a malignant disease caused or characterized by the proliferation of cells which have lost susceptibility to normal growth control. “Malignant disease” refers to a disease caused by cells that have gained the ability to invade either the tissue of origin or to travel to sites removed from the tissue of origin.

As defined herein, “a tumor” is a neoplasm that may either be malignant or non-malignant. Tumors of the same tissue type originate in the same tissue, and may be divided into different subtypes based on their biological characteristics.

As used herein, the term “tumor stage” refers to a measure of the degree of advancement or progression of a tumor. A tumor's stage is determined according to criteria including, for example, the morphology of the cells, morphology of the tissue, whether tumor cells have infiltrated the tissue of origin, whether tumor cells have invaded lymph nodes, and whether distant metastasis has occurred. Clinical staging for many tumors follows the TNM system, but other clinical staging scales adapted to specific diseases are known in the art.

As used herein, the term “degree of disease severity” refers to measure of how advanced a disease is, on a scale from no disease to the worst possible disease. One of skill in the art can place a set of tissue samples representing a disease in order of ascending or descending severity of disease. In order to do so, samples may be compared not only to known standards, but also to each other.

As used herein, the term “difference in biological characteristics” refers to an increase or decrease in a measurable expression of a given biological characteristic. A difference may be an increase or a decrease in a quantitative measure (e.g., amount of a protein or RNA encoding the protein) or a change in a qualitative measure (e.g., location of the protein). Where a difference is observed in a quantitative measure, the difference according to the invention will be at least 10% greater or less than the level in a normal standard sample. Where a difference is an increase, the increase may be as much as 20%, 30%, 50%, 70%, 90%, 100% (2-fold) or more, up to and including 5-fold, 10-fold, 20-fold, 50-fold or more. Where a difference is a decrease, the decrease may be as much as 20%, 30%, 50%, 70%, 90%, 95%, 98%, 99% or even up to and including 100% (no specific protein or RNA present). It should be noted that even qualitative differences may be represented in quantitative terms if desired. For example, a change in the intracellular localization of a polypeptide may be represented as a change in the percentage of cells showing the original localization.

As used herein, the term “substantially matches”, when referring to an expression of a biological characteristic, means that the score assigned to a patients tissue sample for a given polypeptide using a scoring method as described herein is the same (which is defined as not being significantly different using routine statistical tests to within 95% confidence levels) as the score for a tissue sample to which it is being compared for at least that polypeptide. The scoring methods useful in the invention assign a value to every expression characteristic, with each such value actually representing a range of values Since both the patient sample and the standard samples are scored using the same method and the same ranges of values for each class, there will always be a substantial match between a patient sample and one or more tumor or normal samples on the panel, even though the level of expression does not exactly match between the respective samples.

As used herein, the term “non-tumor samples” refers to tissue samples obtained from normal tissue. A sample may be judged a non-tumor sample by one of skill in the art on the basis of morphology or on the basis of molecular characteristics.

As used herein, the term “disease recurrence” refers to the development or emergence of cells of a proliferative disease, such as a tumor, after a treatment that has substantially removed such cells. A disease recurrence may be at the same site as the original disease or elsewhere, but will involve accumulation of cells of the same tissue of origin as in the original disease.

As defined herein, the “efficacy of a drug” or the “efficacy of a therapeutic agent” is defined as ability of the drug or therapeutic agent to restore the expression of diagnostic trait to values not significantly different from normal (as determined by routine statistical methods, to within 95% confidence levels).

As defined herein, “a tissue microarray” is a microarray that comprises a plurality of sublocations, each sublocation comprising tissue cells and/or extracellular materials from tissues, or cells typically infiltrating tissues, where the morphological features of the cells or extracellular materials at each sublocation are visible through microscopic examination. The term “microarray” implies no upper limit on the size of the tissue sample on the array, but merely encompasses a plurality of tissue samples which, in one embodiment, can be viewed using a microscope.

As defined herein a “a sample” is a material suspected of comprising an analyte and includes a biological fluid, suspension, buffer, collection of cells, fragment or slice of tissue. A biological fluid includes blood, plasma, sputum, urine, cerebrospinal fluid, and leukophoresis samples.

The term “donor block” as used herein, refers to tissue embedded in an embedding matrix, from which a tissue sample can be obtained and placed directly onto a slide or placed into a receptacle of a recipient block.

The term “recipient block” as used herein, refers to a block formed from an embedding matrix, having which comprises a plurality of tissue samples; each tissue sample forming the source of a sublocation on a tissue microarray. The relative positions of tissue samples are maintained when the recipient block is sectioned, such that each section comprises sublocations at identical coordinates as any other section from the recipient block.

As defined herein, a “nucleic acid microarray,” a “peptide microarray” or “small molecule” microarray refers to a plurality of nucleic acids, peptides, or small molecules, respectively, respectively that are immobilized on a substrate in assigned (i.e., known) locations on the substrate.

As defined herein, a “database: is a collection of information or facts organized according to a data model which determines whether the data is ordered using linked files, hierarchically, according to relational tables, or according to some other model determined by the system operator. The organization scheme that the database uses is not critical to performing the invention, so long as information within the database is accessible to the user through an information management system. Data in the database are stored in a format consistent with an interpretation based on definitions established by the system operator (i.e., the system operator determines the fields which are used to define patient information, molecular profiling information, or another type of information category). As used herein, a “specimen-linked database” is a database which cross-references information in the database to tissue specimens provided on one or more microarrays, and preferably using codes, such as SNOMED® codes, ICD-9 codes, and/or DSM-IV TR codes.

As defined herein, “a system operator” is an individual who controls access to the database.

As used herein, the term “information management system” refers to a system which comprises a plurality of functions for accessing and managing information within the database. Minimally, an information management system according to the invention comprises a search function, for locating information within the database and for displaying a least a portion of this information to a user, and a relationship determining function, for identifying relationships between information or facts stored in the database.

As defined herein, an “interface” or “user interface” or “graphical user interface” is a display (comprising text and/or graphical information) displayed by the screen or monitor of a user device connectable to the network which enables a user to interact with the database and information management system according to the invention.

As used herein, the term “link” refers to a point-and-click mechanism implemented on a user device connectable to the network which allows a viewer to link (or jump) from one display or interface where information is referred to (“a link source”), to other screen displays where more information exists (a “link destination”). The term “link” encompasses both the display element that indicates that the information is available and a program which finds the information (e.g., within the database) and displays it one the destination screen.

As defined herein, a “browser” is a program which supports the displaying of documents, across a network. Browsers enable accessing linked information over the Internet and other networks, as well as from magnetic disk, CD-ROM, or other memory sources.

As used herein, an “information management system” is a system which comprises searching, organizing, and relationship determination functions.

The term “providing access to at least a portion of a database” as defined herein refers to making information in the database available to user(s) through a visual or auditory means of communication.

As used herein, “through a visual means of communication” includes displaying or providing written text, image(s), or a combination of written and graphical information to a user of the database.

As used herein, “through an auditory means of communication” refers to providing the user with taped audio information, or access to another user who can communication the information through speech or sign language. Written and/or graphical information can be communicated through a printed report or electronically (e.g., through a display on the display of a computer or other processor, through email or other electronic messaging systems, through a wireless communications device, via facsimile, and the like). Access can be unrestricted or restricted to specific subdatabases within the database

The term “report” as used herein refers to a record or summary of the information which may be provided in written, graphical, electronic, or audio form, or combinations of these forms, as described above.

“High throughput techniques” are techniques that evaluate large numbers (at least 10) of samples at a single time.

As used herein, the term “guiding treatment” refers to the process of informing the decision making for the treatment of a disease. As used herein, treatment guidance is based on the comparative levels of expression of one or more biological characteristics (e.g., such as the expression of cell growth-related polypeptides) in a patient's tissue sample relative to the levels of the same biological characteristics(s) in a plurality of normal and diseased tissue samples from individuals for whom patient information, including treatment approaches and outcomes is available.

### Tissue Microarrays

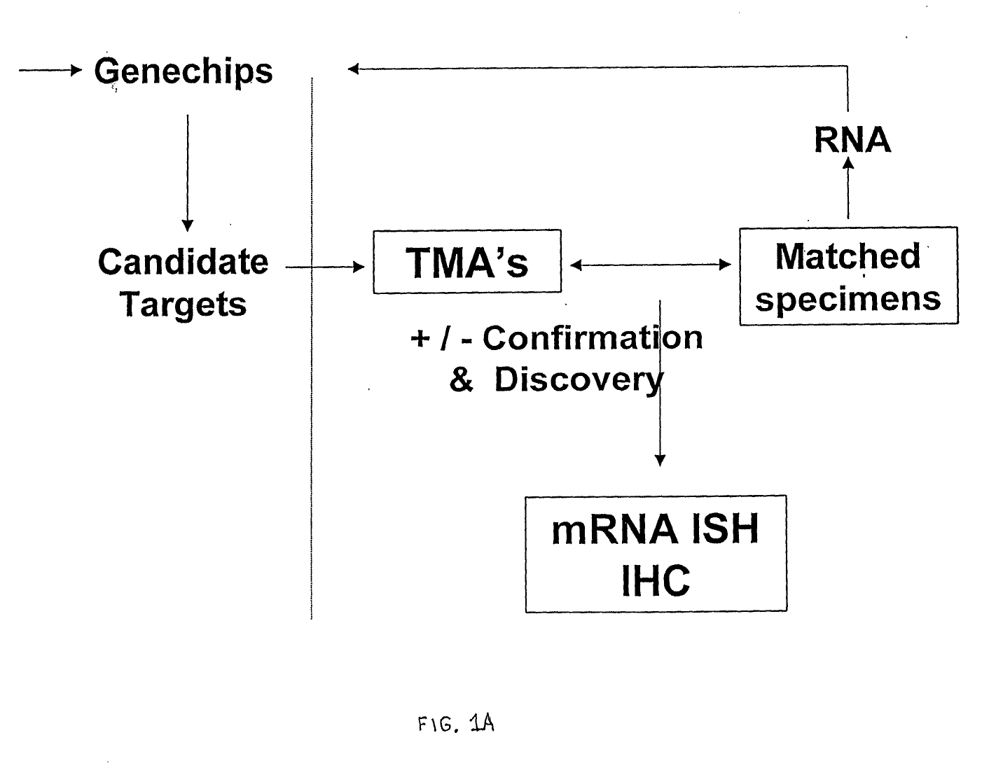

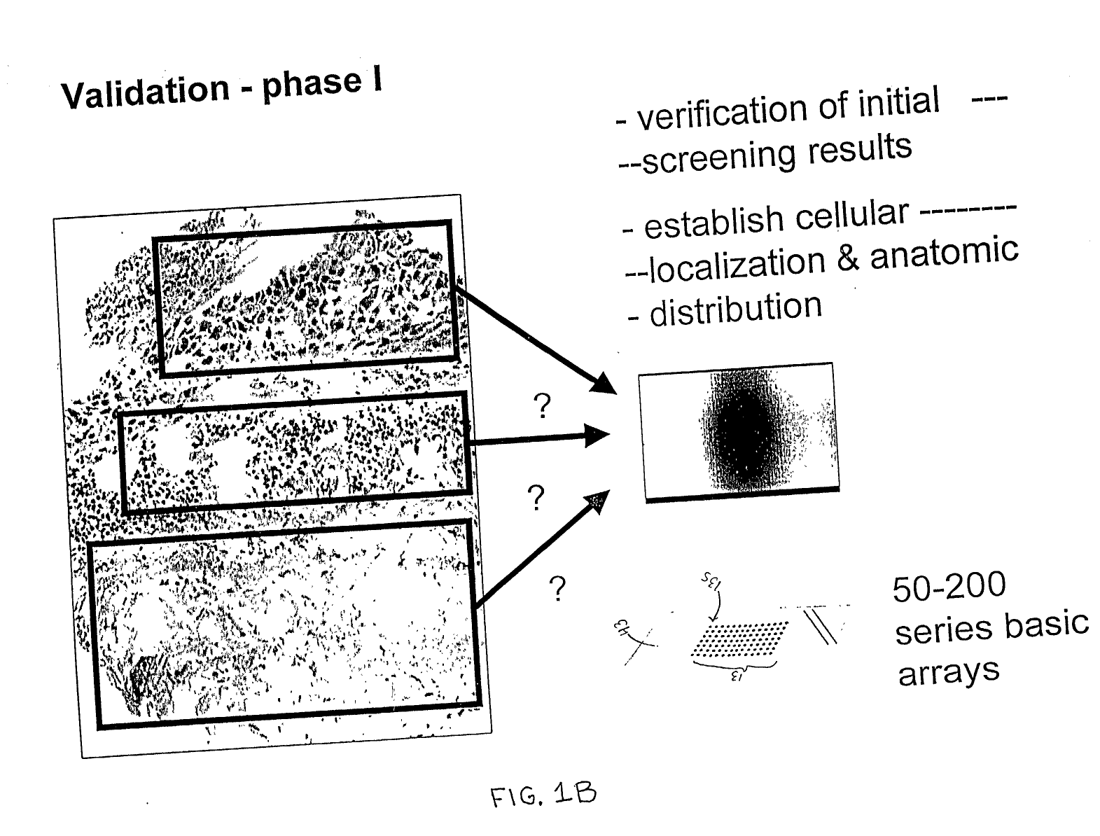

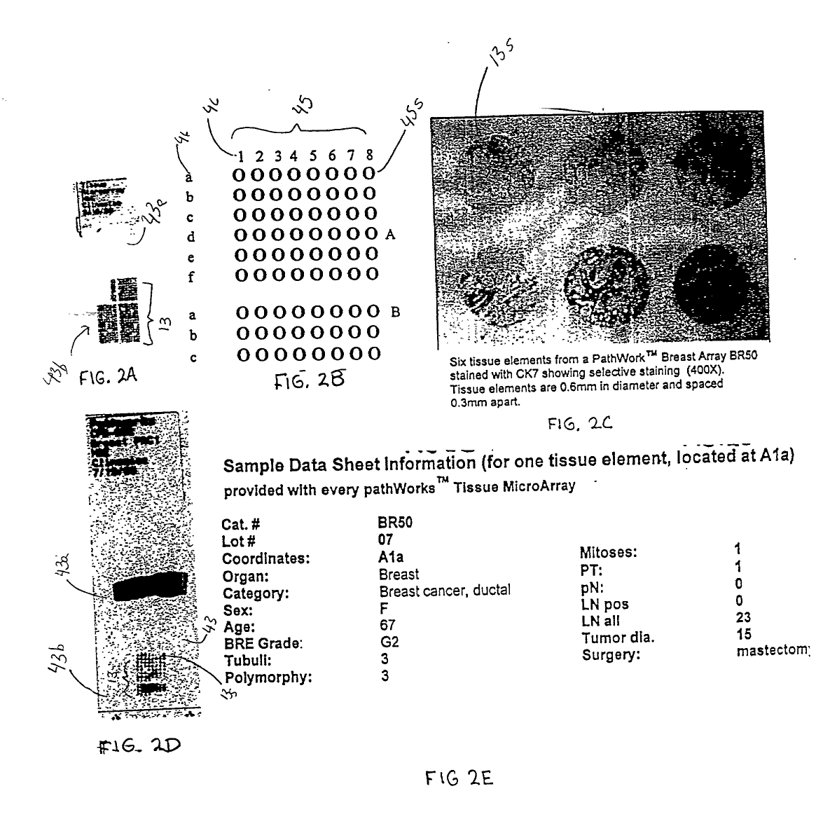

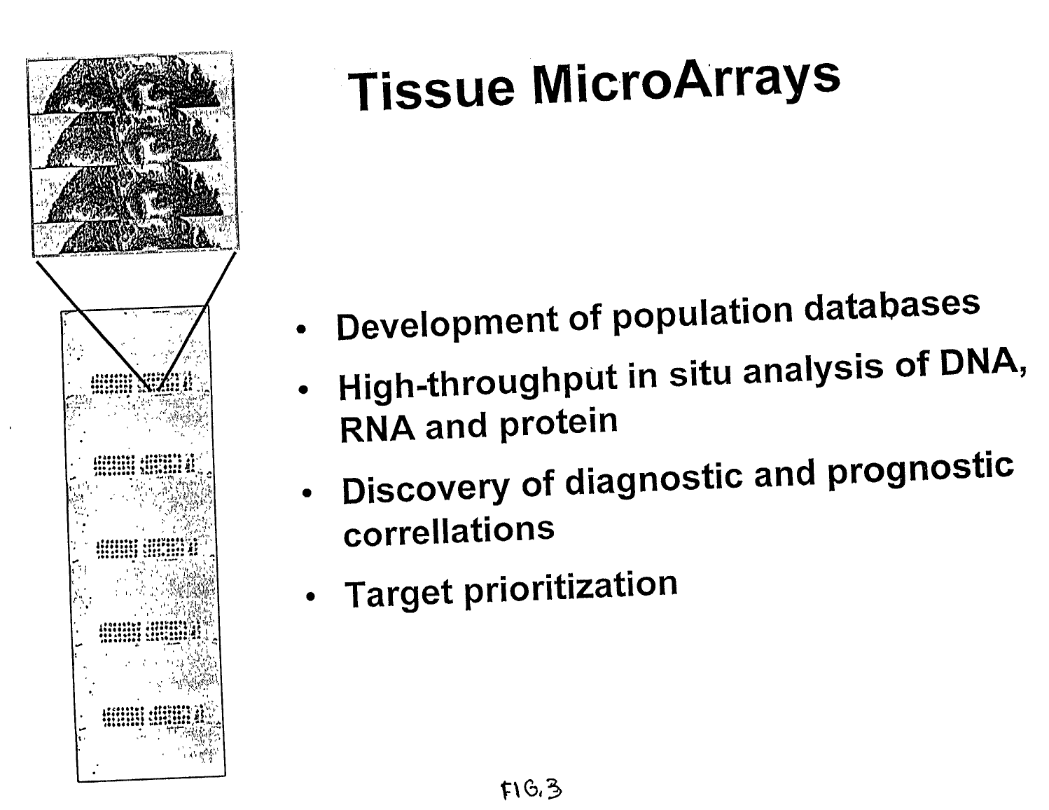

As shown in FIG. 1B, microarrays 13 according to the invention comprise a plurality of sublocations 13s, each sublocation comprising a tissue sample having at least one known biological characteristic (e.g., such as tissue type). In one embodiment, the tissue sample at at least one sublocation 13s has morphological features substantially intact which can be at least viewed under a microscope to distinguish subcellular features (e.g., such as a nucleus, an intact cell membrane, organelles, and/or other cytological features), i.e., the tissue is not lysed (see FIG. 2C and FIG. 3, for example).

In one embodiment of the invention, the microarray comprises a substrate 43 to facilitate handling of the microarray 13 through a variety of molecular procedures. As used herein, “molecular procedure” refers to contact with a test reagent or molecular probe such as an antibody, nucleic acid probe, enzyme, chromagen, label, and the like. In one embodiment, a molecular procedure comprises a plurality of hybridizations, incubations, fixation steps, changes of temperature (from −4° C. to 100° C.), exposures to solvents, and/or wash steps.

In one embodiment of the invention, the microarray substrate 43 is solvent resistant. In another embodiment of the invention, the substrate 43 is transparent. In still another embodiment of the invention, the microarray substrate 43 comprises any of: glass; quartz; fused silica; or other nonporous substrate, plastic, such as polyolefin, polyamide, polyacarylamide, polyester, polyacrylic ester, polycarbonate, polytetrafluoroethylene, polyvinyl acetate, and a plastic composition containing fillers (such as glass fillers), extenders, stabilizers, and/or antioxidants; celluloid, cellophane or urea formaldehyde resins, or other synthetic resins such as cellulose acetate ethylcellulose, or other transparent polymers.

In one embodiment, the microarray substrate 43 is rigid; however, in another embodiment, the substrate 43 is semi-rigid or flexible (e.g., a flexible plastic comprising polycarbonate, cellular acetate, polyvinyl chloride, and the like). In a further embodiment, the substrate 43 is optically opaque and substantially non-fluorescent. Nylon or nitrocellulose membranes can also be used as substrates and include materials such as polycarbonate, polyvinylidene fluoride (PVDF), polysulfone, mixed esters of cellulose and nitrocellulose, and the like.

In one embodiment of the invention, each sublocation 13s of the microarray 13 corresponds to a sublocation 13s on the substrate 43 and each substrate 43 sublocation comprises a tissue stably associated therewith (e.g., able to retain its position relative to another sublocation after exposure to at least one molecular procedure). The size and shape of the substrate 43 may generally be varied. However, preferably, the substrate 43 fits entirely on the stage of a microscope. In one embodiment, the substrate 43 is planar. In one embodiment of the invention, the microarray substrate 43 is 1 inch by 3 inches, 77×50 mm, or 22×50 mm. In another embodiment of the invention, the microarray substrate 43 is at least 10-200 mm×10-200 mm.

In another embodiment of the invention, shown in FIGS. 2A and 2D, the substrate 43 is a “profile array substrate” designed to accommodate a control tissue microarray and a test tissue or cell sample for comparison with the control tissue microarray. In this embodiment, the substrate 43 comprises a first location 43a and a second location 43b. The first location 43a is for placing a test tissue sample, while the second sublocation 43b comprises the microarray 13. This profile microarray substrate 43 allows testing of a test tissue sample to be done simultaneously with the testing of tissue samples on the microarray 13 having at least one known biological characteristic allowing for a side by side comparison of biological characteristics expressed in the test sample with the characteristics of the tissues in the microarray 13. Profile microarray substrates 43 are disclosed in U.S. Provisional Application Serial No. 60/234,493, filed Sep. 22, 2000, the entirety of which is incorporated by reference herein.

### Addressing the Microarray

While the order of sublocations 13s on the microarray 13 is not critical, in a preferred embodiment, the sublocations 13s of the microarray 13 are positioned in a regular repeating pattern (e.g., rows and columns) such that each sublocation 13s can be assigned coordinates relating to its position on the microarray 13 . For example, a sublocation 13s in row 1, column 1, would be assigned the coordinates (1,1), while a sublocation 13s in row 1, column 5 would be assigned coordinates (1,5).

In one embodiment, a microarray locator 45 is provided to enable the user to easily determine the coordinates of a sublocation 13s of interest on the microarray 13. The microarray locator 45 is a template having a plurality of shapes 45s, each shape 45s corresponding to the shape of each sublocation 13s in the microarray 13, and maintaining the same relationships as each sublocation 13s on the microarray 13 (see FIG. 2B, for example). The microarray locator 45 is itself marked by coordinates 46, allowing the user identify the coordinates of sublocation(s) 13s on the microarray 13 by overlaying the microarray locator 45 on top of the microarray 13 and aligning the shapes 45s on the template with the sublocations 13s on the microarray 13. In one embodiment of the invention, the microarray locator 45 is a transparent sheet (e.g., plastic, acetate, and the like). In another embodiment of the invention, the microarray locator 45 is a sheet comprising a plurality of holes, each hole corresponding in shape and location to each sublocation 13s on the microarray 13.

In another embodiment of the invention, substrate 43 itself comprises encoded addressing information at each sublocation 13s on the substrate 43, so that the coordinates of a particular tissue on the microarray 13 can be electronically and remotely determined. For example, in one embodiment of the invention, the substrate 43 is printed on an electrically conductive surface comprising a plurality of address lines. In another embodiment, holes are incorporated into the substrate 43 which may be detected by mechanical or optical means; the holes providing position information (e.g., coordinates) that can be related to information about the tissues at particular sublocations 13s which is stored in the specimen-linked database described further below. Magnetic or other devices can also be incorporated into the substrate 43 to provide a means of identifying the coordinates of selected sublocations 13s on the microarray 13.

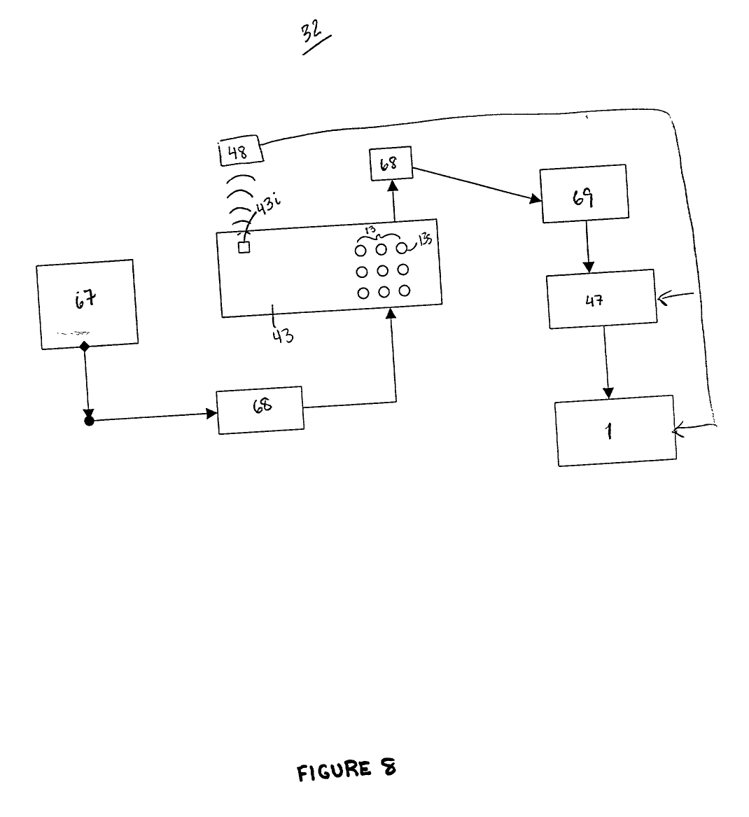

In a further embodiment of the invention, the substrate 43 comprises a location for placing an identifier 43i (e.g., a wax pencil or crayon mark, an etched mark, a label, a bar code, a microchip, or other means for transmitting electromagnetic signals, a radiofrequency transmitter, and the like) (se FIG. 7C and FIG. 8, for example). In one embodiment, the means for transmitting electromagnetic signals communicates with a processor 47 which comprises, or can access, stored information relating to the identity and address of sublocations 13s on the microarray 13, and/or information regarding the individual from whom the tissue was obtained, e.g., such as prognosis, diagnosis, medical history of the patient, family medical history, drug treatment, age of death and cause of death, and the like.

### Sources of Tissue

In one embodiment, the tissues at individual sublocations 13s are from cadavers or patients who have recently died, and/or are from surgical specimens, pathology specimens, or represent “clinical waste” tissue that would normally be discarded from other procedures. In addition to tissue sections, microarrays 13 can also include cells from bodily fluids such as serum, leukophoresis products, and pleural effusions, or cells from cell culture lines (either primary or continuous cell lines).

In one embodiment of the invention, microarray 13 comprises representative tissues from an organism. In one embodiment, the microarray 13 encompasses the “whole body” of one or a plurality of individuals. In another embodiment of the invention, the microarray 13 is a reflection of a plurality of traits representing a particular patient demographic group of interest, e.g., overweight smokers, diabetics with peripheral vascular disease, individuals having a particular predisposition to disease (e.g., to sickle cell anemia, Tay Sachs, severe combined immunodeficiency, and the like).

In another embodiment of the invention, a microarray 13 is provided comprising a plurality of sublocations 13s which represent different stages of a cell proliferation disorder, such as cancer. In one embodiment, the microarray 13 includes metastases to tissues other than the primary cancer site. In still a further embodiment of the invention, the microarray 13 comprises normal tissues, preferably from the same patient from whom the abnormally proliferating tissue was derived. Staged oncology tissue microarrays 13 are described in U.S. Provisional Application Serial No. 60/236,549, filed Sep. 29, 2000, the entirety of which is incorporated by reference herein.

In another embodiment, at least one sublocation 13s comprises cells from a cell line of cancerous cells, either primary or continuous cell lines. Cell lines can be developed from isolated cancer cells and immortalized with oncogenic viruses (e.g., Epstein Barr Virus). Exemplary cell lines which can be used in this embodiment are described in U.S. Provisional Application Serial No.60/236,549, filed Sep. 29, 2000, the entirety of which is incorporated herein by reference

In another embodiment of the invention, the microarray 13 comprises a plurality of sublocations 13s comprising cells from individuals sharing a trait in addition to cancer. In one embodiment of the invention, the trait shared is gender, age, a pathology, predisposition to a pathology, exposure to an infectious disease (e.g., HIV), kinship, death from the same illness, treatment with the same drug, exposure to chemotherapy or radiotherapy, exposure to hormone therapy, exposure to surgery, exposure to the same environmental condition (e.g., such as carcinogens, pollutants, asbestos, TCE, perchlorate, benzene, chloroform, nicotine and the like), the same genetic alteration or group of alterations, expression of the same gene or sets of genes, a disease predisposition, a psychiatric disorder, In another embodiment of the invention, at least one sublocation 13s comprises cells from an individual with an enhanced cancer susceptibility (e.g., a family history of cancer, a patient whose has had cancer previously, or an individual who is exposed to carcinogen(s)).

In one embodiment, the microarray 13 comprises at least one sublocation 13s comprising cancerous cells from a single patient and comprises a plurality of sublocations 13s comprising cells from other tissues and organs from the same patient. In a further embodiment of the invention, each sublocation 13s of the microarray comprises cells from different members of a pedigree sharing a family history of cancer (e.g., selected from the group consisting of siblings, twins, cousins, mothers, fathers, grandmothers, grandfathers, uncles, aunts, and the like). In another embodiment of the invention, the “pedigree microarray” comprises environment-matched controls (e.g., husbands, wives, adopted children, step-parents, and the like).

In a further embodiment of the invention, the microarray 13 comprises at least one sublocation 13s comprising tissue from an individual with a disease other than cancer, or in addition to cancer (e.g., including, but not limited to: a blood disorder, blood lipid disease, autoimmune disease, bone or joint disorder, a cardiovascular disorder, respiratory disease, endocrine disorder, immune disorder, infectious disease, muscle wasting and whole body wasting disorder, neurological disorders (including both the central nervous system and peripheral nervous system), skin disorder, kidney disease, scleroderma, stroke, hereditary hemorrhage telangiectasia, disorders associated with diabetes, hypertension, diabetes, manic depression, depression, borderline personality disorder, anxiety, schizophrenia, Gaucher disease, cystic fibrosis and sickle cell anemia, liver disease, pancreatic disease, eye, ear, nose and/or throat disease, diseases affecting the reproductive organs, gastrointestinal diseases, including diseases of the colon, diseases of the spleen, appendix, gall bladder, and the like). For further discussion of human diseases, see Mendelian Inheritance in Man: A Catalog of Human Genes and Genetic Disorders by Victor A. McKusick (12th Edition (3 volume set) June 1998, Johns Hopkins University Press, ISBN: 0801857422), the entirety of which is incorporated herein.

In another embodiment, microarrays are provided which comprise tissue samples from patients suffering from a neurodegenerative disease, i.e., a disease which causes progressive cell damage of neurons within the central nervous system (CNS) leading to loss of neuronal activity and cell death. Neurodegenerative diseases encompassed within the scope of the invention encompass chronic neurodegenerative diseases, including, but not limited to: AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supra-nucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi-system disorder); demyelinating core disorders, such as multiple sclerosis, acute transverse myelitis; and disorders of the motor unit such as neurogenic muscular atrophies (anterior horn cell degeneration, such as amyotrophic lateral sclerosis, primary lateral sclerosis infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis Hallerrorden-Spatz disease; and Dementia pugilistica, diabetic peripheral neuropathy. (see, e.g., Berkow et al, eds., The Merck Manual, 16th edition, Merck and Co., Rahway, N. J., 1992, which reference, and references cited therein, are entirely incorporated herein by reference). Acute neurodegenerative diseases are also encompassed within the scope of the invention, such as conditions arising from stroke, schizophrenia, cerebral ischemia resulting from surgery and epilepsy as well as hypoglycemia and trauma resulting in injury of the brain, peripheral nerves or spinal cord, and the like.

In a further embodiment, microarrays are provided which comprise tissue samples from patients who have a neuropsychiatric disorder. Such disorders include, but are not limited to, mental retardation, a learning disorder, a motor skills disorder, a communication disorder, a pervasive developmental disorder (e.g., autism, childhood disintegrative disorder, Rett's disorder), attention deficit and disruptive behavior disorders, eating disorders, tic disorders, elimination disorders (encopresis, enurisis), selective mutism, separation anxiety disorder, reactive attachment disorder of infancy or early childhood, delirium, dementia, amnestic disorders, cognitive disorders, catatonic disorder, personality change disorder, substance dependence or other substance induced disorders (e.g., a drug or alcohol abuse related disorder), schizophrenia (e.g., catatonic, disorganized, paranoid, residual, undifferentiated), schizophreniform disorder, delusional disorder, brief psychotic disorder, shared psychotic disorder, psychotic disorder due to a general medical condition (e.g., delusions, hallucinations), a substance-induced psychotic disorder, mood episodes (major depressive episode, hypomanic episode, manic episode, mixed episode), depressive disorders, bipolar disorders, acute stress disorder, agoraphobia, anxiety disorder, obsessive-compulsive disorder, panic disorder with or without agoraphobia, postraumatic stress disorder, obsessive-compulsive disorder, body dysmorphic disorder, conversion disorder, hypochondriasis, and other somatoform disorders, a dissociative disorder, a sexual or gender identity disorder, an eating disorder (e.g., anorexia, bulimia nervosa), a sleep disorder, kleptomania, pyromania, pathological gambeling, intermittent explosive disorder, an Axis II personality disorder (each disorder as classified using DSM-IV criteria).

In one embodiment, sets of microarrays 13 are provided representing multiple individuals with approximately 30,000 tissue specimens covering at least 5, 10, 15, 20, 25, 30, 40, or 50, different disease categories, including, but not limited to, any of the disease categories identified above.

Although in a preferred embodiment of the invention the microarrays 13 comprise human tissues, in one embodiment of the invention, abnormally proliferating tissues from other organisms are arrayed. In one embodiment, the microarray 13 comprises tissues from non-human animals (e.g., mice) which have either spontaneously developed cancer or who have received transplants of tumor cells. In one embodiment, the microarray 13 comprises multiple tissues from such a non-human animal. In another embodiment of the invention, the microarray 13 comprises tissues from non-human animals which have spontaneously developed cancer or who have received transplants of tumor cells, and which have been treated with a cancer therapy (e.g., drugs, antibodies, protein therapies, gene therapies, antisense therapies, and the like).

In still a further embodiment of the invention, tissues from a non-human animal genetically engineered to over express or under express desired genes are provided. In one embodiment, a microarray 13 is provided comprising tissues from non-human animals expressing different doses of the same cell proliferation gene or tumor suppressor gene. In still a further embodiment, a microarray 13 is provided comprising a plurality of cell lines (normal and/or cancer cell lines) which have been genetically engineered to express cell proliferation genes or tumor suppressor genes or modified forms of such genes. In this embodiment, cells may stably or transiently transfected cell lines, or genetically engineered tumors (e.g., such as by infection with a recombinant retroviral vector).

In one embodiment, the tissue microarray 13 comprises tissues from different recombinant inbred strains of individuals (e.g., mice). In a further embodiment, tissues from humans comprising a characterized haplotype are arrayed (e.g., a particular grouping of HLA alleles).

### Construction of Tissue Microarrays

Tissue microarrays 13 according to the invention are generated by obtaining donor tissues from any of the tissue sources described above, embedding these tissues, and obtaining portions of the embedded tissue for placement in a “recipient block,” a block of embedding matrix which can subsequently be sectioned, each section being placed on any of the substrates described above. Therefore, in one embodiment, the invention encompasses recipient blocks for forming any of the microarrays 13 disclosed above.

### Embedding Tissues: Forming Donor Blocks

In one embodiment of the invention, tissues are obtained and either paraffin-embedded, plastic-embedded, or frozen. When paraffm-embedded tissues are used, a variety of tissue fixation techniques can be used. Examples of fixatives, include, but are not limited to, aldehyde fixatives such as formaldehyde, formalin or formol, glyoxal, glutaraldehyde, hydroxyadipaldehyde, crotonaldehyde, methacrolein, acetaldehyde, pyruvic aldehyde, malonaldehyde, malialdehyde, and succinaldehyde; chloral hydrate; diethylpyrocarbonate; alcohols such as methanol and ethanol; acetone; lead fixatives such as basic lead acetates and lead citrate; mercuric salts such as mercuric chloride; formaldehyde; dichromate fluids; chromates; picric acid, and heat.

Tissues are fixed until they are sufficiently hard to embed. The type of fixative employed will be determined by the type of molecular procedure being used, e.g., where the molecular characteristic(s) being examined include the expression of nucleic acids, isopentane, or PVA, or another alcohol-based fixative is preferred, paraffin is preferred for performing immunohistochemistry, in situ hybridization, and in general, for tissues which are going to be stored for long periods of time. When cells are obtained from plasma, the cells may be snap frozen. OCT embedding is optimal for morphological evaluations.

Embedding media encompassed within the scope of the invention, includes, but is not limited to paraffin or other waxes, plastic, gelatin, agar, polyethlene glycols, polyvinyl alcohol, celloidin, nitrocelluloses, methyl and butyl methacrylate resins or epoxy resins. Water-insoluble embedding media such as paraffin and nitrocellulose require that specimens be dehydrated in several changes of solvent such as ethyl alcohol, acetone, xylene, toluene, benzene, petroleum, ether, chloroform, carbon tetrachloride, carbon bisulfide, and cedar oil. or isopropyl alcohol prior to immersion in a solvent in which the embedding medium is soluble. Water soluble embedding media such as polyvinyl alcohol, carbowax (polyethylene glycols), gelatin, and agar, can also be used.

In one embodiment, tissue specimens are freeze-dried by deep freezing in plastic tissue cassettes and storing them at −80-70° C., such as in liquid nitrogen. In one embodiment, the tissues are then covered with a cryogenic media, such as OCT®, and kept at −80-70° C., until sectioned. Examples of embedding media for frozen tissues include, but are not limited to, OCT, Histoprep®, TBS, CRYO-Gel®, and gelatin, to name a few. In another embodiment, a tissue freezing aerosol may be used to facilitate embedding of the donor frozen tissue block. An example of a freezing aerosol is tetrafluoroethane 2.2. Other methods known in the art may also be used to facilitate embedding of a tissue sample.

### Forming the Recipient Block

In one embodiment, microarrays according to the invention are constructed by coring holes in a recipient block comprising an embedding substance (e.g., paraffin, plastic, or a cryogenic media) and placing a tissue sample from a donor block in a selected hole. Holes can be of any shape and size, but are preferably made in a regular pattern. In one embodiment of the invention, the hole for receiving the tissue sample is elongated in shape. In another embodiment, the hole is cylindrical in shape.

While the order of the donor tissues in the recipient block is not critical, in some embodiments, donor tissue samples are spatially organized. For example, in one embodiment, donor tissues represent different stages of disease, such as cancer, and are ordered from least progressive to most progressive (e.g., associated with the lowest survival rates). In another embodiment, tissue samples within a microarray 13 will be ordered into groups which represent the patients from which the tissues are derived. For example, in one embodiment, the groupings are based on multiple patient parameters that can be reproducibly defined from the development of molecular disease profiles. In another embodiment, tissues are coded by genotype and/or phenotype. Tissue samples on the microarray 13 can additionally be arranged according to treatment approach, treatment outcome, or prognosis, or according to any other scheme that facilitates the subsequent analysis of the samples and the data associated with them.

The recipient block can be prepared while tissue samples are being obtained from the donor block. However, in one embodiment, the recipient block is prepared prior to obtaining samples from the donor block, for example, by placing a fast-freezing, cryo-embedding matrix in a container and freezing the matrix so as to create a solid, frozen block. The embedding matrix can be frozen using a tissue freezing aerosol such as tetrafluorethane 2.2 or by any other methods known in the art. The holes for holding tissue samples can be produced by punching holes of substantially the same dimensions into the recipient block as those of the donor frozen tissue samples and discarding the extra embedding matrix.

Information regarding the coordinates of the hole into which a tissue sample is placed and the identity of the tissue sample at that hole is recorded, effectively addressing each sublocation 13s on the microarray 13. In one embodiment of the invention, data relating to any, or all of, tissue type, stage of development or disease, individual of origin, patient history, family history, diagnosis, prognosis, medication, morphology, concurrent illnesses, expression of molecular characteristics (e.g., markers), and the like, is recorded and stored in a database, indexed according to the location of the tissue on the microarray 13. Data can be recorded at the same time that the microarray 13 is formed, or prior to, or after, formation of the microarray 13.

The coring process can be automated using core needles coupled to a motor or some other source of electrical or mechanical power. In one embodiment of the invention, a microarray 13 is generated using a Beecher Instruments Tissue Microarrayer (Beecher Instruments, Silver Springs, Md.), or an automated microarray 13 as described in U.S. Pat. No. 6,103,518, the entirety of which is incorporated by reference herein. These devices basically consist of a turret containing two hollow core borer needles, one larger than the other, mounted on a platform with a spring mechanism. The smaller needle removes a core from the recipient block while a larger needle removes a core of tissue from the donor tissue block by means of stylet(s). The stylet is inserted into the smaller needle thereby injecting the donor tissue core into the hole made in the recipient block, while the same, or another, stylet is used to remove embedding media remaining in the smaller core borer needle, permitting its reuse. The stylets described in U.S. Pat. No. 6,103,518, are designed primarily for use with paraffin tissue sections. Stylets which are designed especially for use in arraying frozen tissues are described in U.S. patent application Ser. No. _____, filed Feb. 8, 2000, entitled “Stylet For Use With Tissue Microarrayer and Molds,” Attorney Docket No. 5568/1070 and U.S. Design application Ser. No. 29/131,964 filed Oct. 31, 2000 (the entireties of which are incorporated by reference herein).

In one embodiment of the invention, large formats microarrays 13 are provided which comprise at least one sublocation greater in at least one diameter than 0.6 mm. In another embodiment, at least one sublocation comprises a heterogeneously expressed biomolecule which is expressed in less than 80% of cells in a given tissue type and which is diagnostic of a disease. In a further embodiment of the invention, the large format microarray 13 comprises at least one sublocation 13s comprising at least two different cell types or cellular material (e.g., any of abnormally proliferating cells (e.g., cancerous cells), stromal cells, extracellular matrix, necrotic cells and apoptotic cells).

Large format microarrays 13 can be used alone or in conjunction with small format microarrays 13 (microarrays 13 in which individual sublocations 13s are less than 0.6 mm in diameter). In one embodiment of the invention, a large format microarray 13 is used in conjunction with a small format microarray 13 derived from the same patient's tissue sample. In this embodiment, the large format microarray 13 can be used to demonstrate that the biological characteristics of the smaller sublocations of the small format microarray 13 are representative of the biological characteristics within a larger sample. Methods of constructing large format microarrays 13 are disclosed in U.S. patent application Ser. No. ______, filed Feb. 8, 2001, entitled, “Large Format Microarrays” (Attorney Docket No. 5568/1050), the entirety of which is incorporated by reference herein.

Other methods of generating microarrays 13 are described in U.S. Provisional Application Number 60/213,321, the entirety of which is incorporated by reference herein, and in WO 99/44062 and WO 99/44062, incorporated entirely by reference herein, and are encompassed within the scope of the instant invention.

### Tissue Information System for Accessing Organizing and Displaying Information Regarding Tissue Microarrays

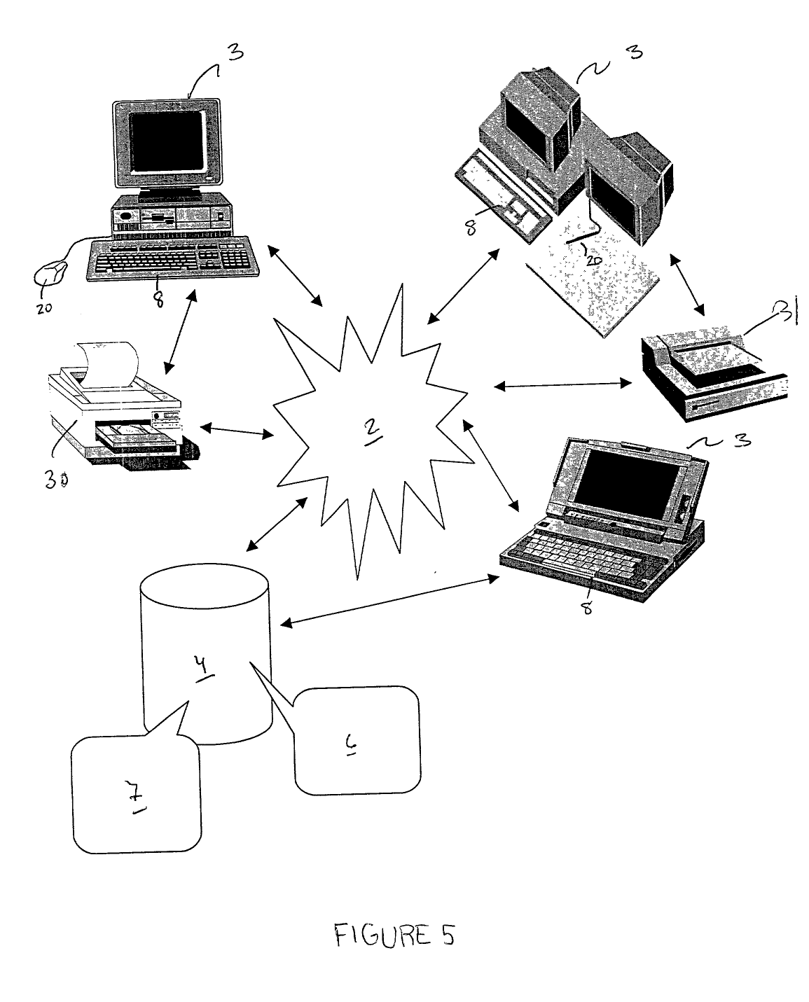

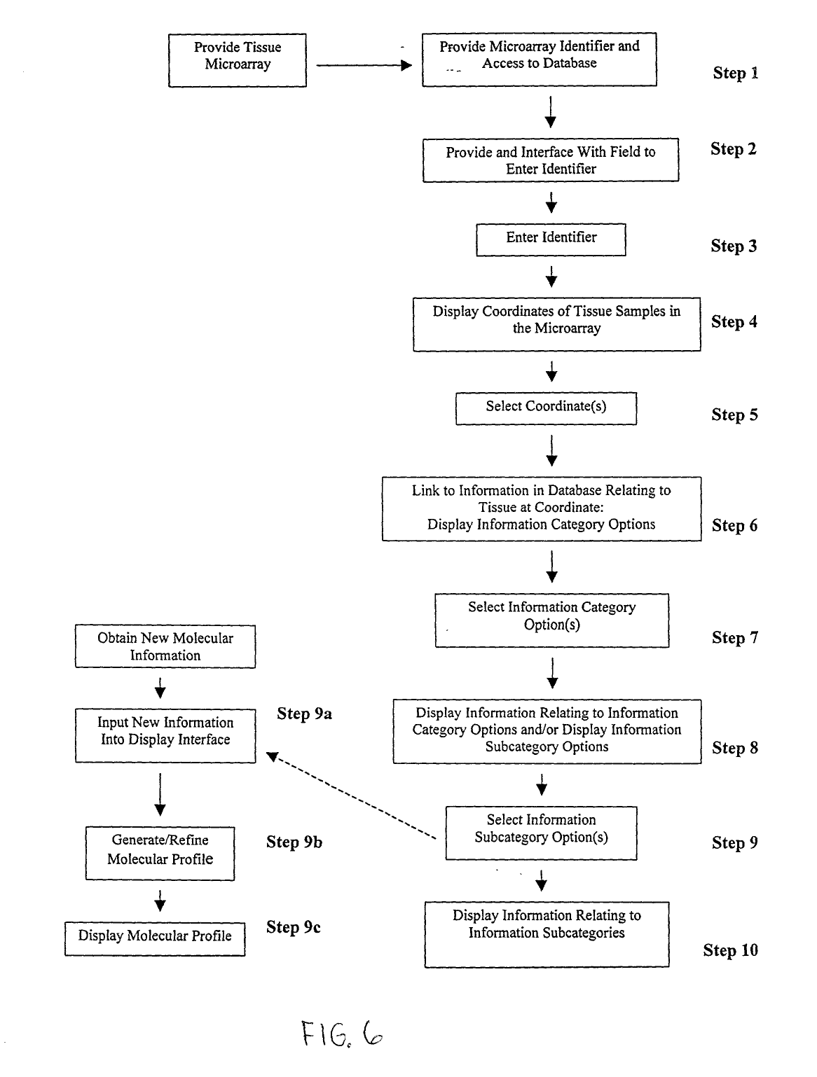

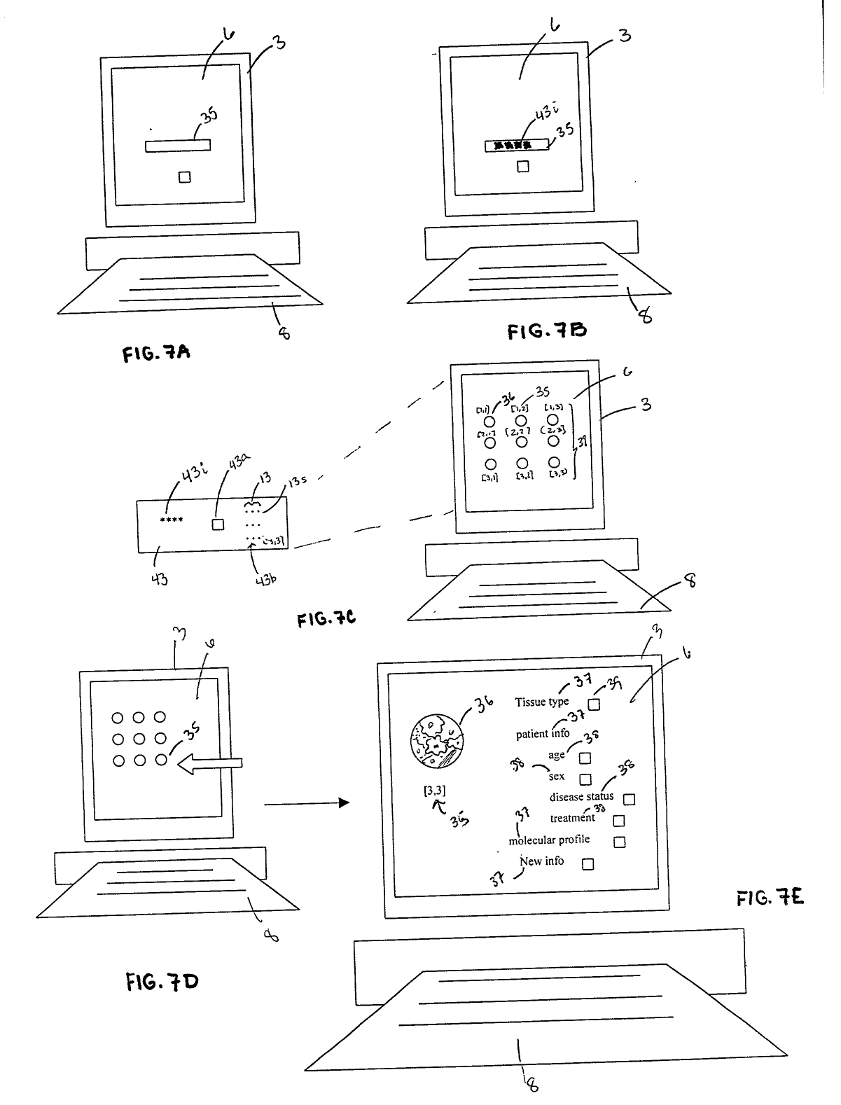

The invention provides a tissue information system 1 (shown in FIG. 5) for accessing, organizing, and displaying information relating to tissue microarrays 13. The tissue information system 1 comprises at least one user device 3 connected to a network 2. In one embodiment, the network is wide area network (WAN) to which the at least one user device 3 is directly connected. However, in another embodiment, user device 3 is connected to a WAN indirectly through a local area network (e.g., via a proxy server).

Because the user device 3 is connected to the network 2, individual steps of accessing, organizing, and displaying can be performed on one, or a plurality, of user devices 3 at different physical locations. Thus, in one embodiment of the invention, one or more tissue microarrays are each screened at physically distant locations, for example, in different laboratories, hospitals, or companies, and the information obtained from the microarrays screened at each location is correlated with tissue information included within the specimen-linked database 5. Multiple users can both access and add to information within the database 5.

Accessing the system 1 through the user device 3 results in an interface 6 being displayed on a display of the device 3. The interface 6 comprises at least one link to a specimen-linked database 5 which comprises tissue information. In one embodiment, the database 5 is also coupled to an information management system (IMS) 7 which comprises both information search functions and relationship determination functions for presenting information to the user in a useable form.

The device 3 comprises a processor and further includes processor readable storage media or electronic memory that can be accessed by the processor. Processor media includes volatile and nonvolatile media, such as RAM, ROM, EPROM, flash memory, CD-ROM, digital versatile disks (DVD), optical storage media, cassettes, tape, discs, and the like. The device 3 can further include multimedia rendering functions by including audio and video components (not shown). In one embodiment, the device 3 also comprises an operating system (e.g., such as Microsoft Windows, UNIX X-Windows, or Apple MacIntosh System) and one or more application programs, including an Internet or Web browser, such as Microsoft's Internet Explorer™, or Netscape® (see, as described in Internet Starter Kit by Adam Engst, Corwin Low and Michael Simon, Second Edition, Hayden Books, 1995, the entirety of which is incorporated by reference herein).

Web browsers enable a user of the user device 3 to click on portions of an interface 6 displayed on the display of a user device 3, triggering a response by the system 1. In one embodiment, the response by the system I is to download and display tissue information on the interface 6 or to provide links to sources of tissue information. In addition to browsers, other networking systems can be included in the tissue information system 1, such as routers, peer devices, common network nodes, modems, and the like.

Suitable devices 3 connectable to the network 2 which are encompassed within the scope of the invention, include, but are not limited to, computers, laptops, microprocessors, workstations, personal digital assistants (e.g., palm pilots), mainframes, wireless devices, and combinations thereof. In one embodiment, the device 3 comprises a text input element 8, such as a key board or touch pad, enabling the user to input information into the system 1. In another embodiment, navigating devices 20 are coupled to the device 3 to allow the user to navigate an interface 6. Navigating devices 20 include, but are not limited to, a mouse, light pen, track ball, joystick(s) or other pointing device.

In one embodiment, the system 1 comprises at least one server 4. The server 4 provides access to one or more data storage media such as hard disks or hard disk arrays. In one embodiment, the server 4 maintains the database 5 on one of these hard disks. In one embodiment, the server 4 comprises one or more applications, including the IMS 7, which permits a user to access information within the database 5, as well as to implement programs for determining relationships between data in the database 5 and tissues on the microarray 13. In another embodiment, another application program is provided which implements the search function of the IMS 7. In a further embodiment, application programs which retrieve records also perform user-defined operations on the records (e.g., such as creating folders in which to store records of particular interest to a user). Applications programs ordinarily are written in a general purpose host programming language, such as C<+++>; however, also include user-defined statements written in a relational query language such as SQL.

In further embodiments of the invention, the system 1 comprises information out put modules 30 (e.g., printers) for outputting and reporting information from the database 5. The system can also comprise information input modules 31 (e.g., scanners), for receiving information from a user, such as scanned data.

In still another embodiment of the invention, a molecular profiling system 32 (such as the one shown in FIG. 8) is provided which is connectable to the device 3. In one embodiment, molecular profiling data is automatically inputted into the database 5, and a user accessing the system 1 has immediate access to this data.

### Specimen-Linked Database

Information within the specimen-linked database 5 is dynamic, being added to and refined as additional users access the database 5 through the system 1. In one embodiment, inputted information at least comprises information relating to the analyses of the tissue microarrays 13 described above and the database 5 organizes this information according to a data model. Data models are known in the art and include flat file models, indexed file models, network data models, hierarchical data models, and relational data models. Flat file models store data in records composed of fields and are dependent upon the particular applications comprising the IMS 7, e.g., if the flat file design is changed, the applications comprising the IMS 7 must also be modified. Indexed file systems comprise fixed-length records composed of data fields and indexes which group data fields according to categories.

A network data model also comprises fixed-length records composed of data fields which are indexed according to categories. However, network data models provide record identifiers and link fields to connect records together for faster access. Network data models further comprise pointer structures which provides a shorthand means of identifying linked records. Hierarchical data models comprise fixed-length records composed of data fields, indexes, record identifiers, link fields, and pointer structures, but further represent the relationship of different records in a database in a tree structure.

In contrast, relational data models comprise tables comprising columns and rows of data elements or attributes. Attributes provide information about the different facts stored within the database 5. Columns within the table comprise attributes of the same data type (e.g., in one embodiment, all information relating to patient X's drug exposure), while each row of the table represents a different relationship (e.g., row one, representing dosage, row two representing efficacy, row three representing safety). As with network data models, and hierarchical data models, relational database models link related information within the database.

Any of the data models described above can be used to organize information within the database 5 into information categories to facilitate access by a user of the tissue information system 1. In a preferred embodiment, a system operator, i.e., the user who provides access to the tissue information system to other users, determines the parameters which define a particular information category recognized by a particular data model.

For example, in one embodiment, the system operator determines the fields that are used to define the information category “drug exposure.” In this embodiment, the system operator may determine that these fields should include: “types of drugs to which the patient was exposed;” “frequency of exposure;” “dose at each exposure;” “physiological response to exposure;” “tests used to measure physiological responses;” “molecular response to exposure;”; “tests used to measure molecular responses,” and the like. Similarly, the system operator may determine that fields which define the information category “medical history of a patient” should encompass all information obtained by health care workers at any time during the patient's life as well as information relating to tests performed by health care workers, or should encompass only selected portions of such records. It should be obvious to those of skill in the art that information categories determined by the system operator can overlap in the types of information contained within them. For example, information relating to medical history could include information relating to a patient's drug exposure. In one embodiment, therefore, the database 5 further comprises links between different information categories which comprise areas of overlap.

The parameters defined by the system user are included within a database dictionary portion of the database 5 and in one embodiment, a user other than the system operator can access the database dictionary on a read-only basis to determine what parameters were used to define a particular information category. In another embodiment of the invention, a user of the system can request that additional parameters be included in the definition of an information category, and, subject to the approval of the system operator, the definition of the information category can be modified as the database expands. In a further embodiment, the database 5, for example, as part of the dictionary can include a table comprising word equivalents to facilitate searching by the IMS-7.

In one embodiment, new information inputted into the system 1 is stored within a temporary database and is subject to validation by the system operator prior to its inclusion in the portion of the database 5 to which all users of the system have access to. FIG. 12 illustrates an example of a quality control procedure to validate data within the specimen linked database 5 In another embodiment, data within the temporary database, is fully able to be accessed and compared to information within the specimen-linked database 5; however, users of the system 1 are alerted to the fact that data within the temporary database has not necessarily been validated (e.g., repeated or evaluated as to quality). In this embodiment, the information categories included within the temporary database can include information relating to the time and date on which the new information was inputted into the system 1.

In one embodiment of the invention, information within information categories is derived from an analysis of any of the tissue microarrays described above. For example, in one embodiment, the database 5 comprises information reflective of “whole body microarrays” which have been evaluated by user(s). In this embodiment, information included within the database encompasses information relating to the types of tissue on the microarray and relating to biological characteristics of the tissue source (e.g., such as patient information). In another embodiment, the database 5 comprises information including, but not limited to, the sex and age of the tissue source, underlying diseases affecting the tissue source, the types of drugs or other therapeutic agents being taken by the tissue source, the localization of the drugs and agents in the different tissues of the microarray, and the effects of the drugs and agents on the different tissues of the microarray, environmental conditions to which the tissue source has been, and is being exposed to, as well as the lifestyle of the tissue source (e.g., moderate or no exercise, alcohol, tobacco consumption, and the like), cause of death, and age of death (if appropriate).

In further embodiments of the invention, information from a plurality of microarrays 13 is used to create the database 5, providing information relating to populations of individuals (e.g., such as demographic and/or epidemiological information). In one embodiment, information relating to microarray(s) 13 comprising at least one disease tissue sample (e.g., a tissue sample expressing biological characteristics associated with disease) is included within the database 5. In one embodiment, this information relates to biological characteristics which define different stages of the disease (e.g., biological characteristics which are associated with different stages of cancer). In another embodiment, information relating to the biological characteristics of normal tissues from the same or different patients is also included within the database 5. In a further embodiment, patient information relating to the tissue sources of tissues at different sublocations 5 on microarray(s) 13 is included within the database, providing information such as gender, age, underlying diseases, family information, cause and time of death if appropriate, information relating to treatment with drugs or other therapeutic agents (e.g., such as protein or nucleic acid-based therapeutic agents), and/or exposure to chemotherapy, radiotherapy, surgery, environmental conditions, and the like.

While in one embodiment, the database 5 comprises information relating to human tissues, in another embodiment, the database 5 also includes information from non-human tissues (e.g., animals, plants, and/or genetically engineered animals or plants). For example, in one embodiment, the database 5 includes information relating to the biological characteristics of non-human tissues which have been exposed to any of drugs, antibodies, protein therapies, gene therapies, antisense therapies, and the like. In some embodiments, the biological characteristics of tissues from non-human individuals which have been genetically engineered to over express or under express desired genes are included within the database 5. In a further embodiment, information within the database 5 also includes information from cell lines (normal and/or cancer cell lines) which have been genetically engineered to express desired genes (e.g., cell proliferation genes or tumor suppressor genes or modified forms of such genes).

In one embodiment, the database comprises information relating to tissues from different recombinant inbred strains of individuals (e.g., mice). Such information includes, but is not limited to, the allele carried at one or more loci, haplotype information, and information relating to the expression of one or more proteins encoded by these loci. In a further embodiment, information relating to diseases associated with particular alleles or haplotypes are further included within the database.

In one embodiment, the database 5 comprises molecular profiling data (i.e., information relating to the expression of one or more biomolecules). In one embodiment, molecular profiling data is obtained from any of normal tissue, diseased tissue (including tissues at different stages of disease), different developmental stages from one or more different types of organisms, and from tissues which have been genetically engineered to include different doses or altered forms of gene(s). Molecular profiling data from whole body microarrays as well as microarrays reflecting populations of individuals can also be included within the database 5. In one embodiment, molecular profiling data includes the expression pattern of a plurality of genes expressed during cancer, a patient having one or more of an autoimmune disease, a neurodegenerative disease (either chronic or acute), a neuropsychiatric disorder, a respiratory disorder, a skin disorder, an endocrine disorder, and the like. In another embodiment, molecular profiling data includes data relating to genes expressed during selected physiological processes. In still another embodiment, molecular profiling data includes data relating to the expression of genes within a pathway during a normal or disease state.

While in one embodiment, information within the database 5 is obtained from tissues provided on the microarrays 13 described above, tissue information can also be obtained from a variety of other sources, such as test samples assayed alongside the tissue microarrays 13 (e.g., using profile array substrates), or test samples which have been assayed independently of tissue microarrays 13, or tissue samples from cell lines, or tissue panels from living patients or from archived tissues, and the like. Information relating to nucleic acid microarrays, protein, polypeptide, peptide, and other biomolecule arrays can also be included within the database, irrespective of whether information from a corresponding tissue microarray 13 has also been obtained. As used herein, although the database is described as being “specimen linked” the database can also include data unrelated to specific test specimens.

In one embodiment, the specimen linked database 5 can be organized to facilitate information retrieval by the IMS 7 by providing a plurality of “subdatabases”, each of which comprises information relating to a particular category of tissue information. For example, in one embodiment, the subdatabases comprise information relating to any of: oncology, cardiovascular diseases, respiratory diseases, renal diseases, gastrointestinal diseases, liver diseases, metabolic diseases, endocrine diseases, infectious diseases, inflammatory diseases, musculoskeletal diseases, neurological diseases, dermatological diseases, gynecological diseases, and urological diseases.

In another embodiment, subdatabases are restricted to particular types of information and include, but are not limited to, sequence subdatabases, protein structure subdatabases, chemical formula/structure subdatabases, expression pattern subdatabases (e.g., providing information relating to the expression of genes in different tissues), information relating to drug targets and drug leads (e.g., including, but not limited to information relating to compound toxicity, side effects, efficacy, metabolism, drug interactions), as well as literature subdatabases, medical history subdatabases, demographic information subdatabases, and the like.

In one embodiment of the invention, data within the database 5 is defined using SNOMED® Clinical Term™. For example, different clinical concepts (e.g., cardiovascular disease, neurodegenerative disease, autoimmune disease, cancer, reproductive disease, neuropsychiatric diseases) are assigned unique concept identifiers which are represented within a “Concept Table” within the database 5. Concepts can be defined by codes, such that a string of codes can be used to cross reference data from a plurality of databases and subdatabases.

In a further embodiment, the database 5 stores uncompressed raw data files, such as for example, microscopy and histological data obtained from the tissues. In this embodiment, the database 5 is of a magnitude which enables storage of memory intensive files, and the network 2 connection enables high speed (T-1, T-3 or higher) transmission of the data to the user. In still another embodiment of the invention, data relating to an image of the test tissue is stored within the database 5, and the image can be displayed by the user upon accessing the database 5.

Thus, as described above, the specimen-linked database 5 according to the invention makes information available concurrently from a number of different sources to enable a user to practice “genomic medicine,” i.e., to develop diagnostic and treatment modalities based not only on the physiological responses of a patient, but also on the biomolecular responses of a patient. As illustrated in the table below, in one embodiment, a genomic medicine database is provided which comprises a plurality of subdatabases, including, but not limited to, a patient information subdatabase, a medical information subdatabase, a pathology information subdatabase, and a genomic information subdatabase. As can be seen from the table, information in one database may overlap (i.e., be repeated) in another database. For example, a pathology subdatabase can included molecular information relating to a particular disease, just as can a genomics database, but may also include additional information, such as information identifying the correlation between a particular marker and a morphological characteristic.

Genomic Medicine Database

| Patient | Medical | Pathology | Genomic |
|---|---|---|---|
| Information | Information | Information | Information |
| Subdatabase | Subdatabase | Subdatabase | Subdatabase |
| Demographics | Diagnosis | Diagnosis | DNA |
| Life style | Other conditions | Histology | Protein |
| Epidemiology | Concurrent Illness | Clinical Data | mRNA |
| Family History | Medications | Molecular Markers | |
| | Outcome Survival | | |

### Search And Relationship Determination System For Accessing Tissue Information From The Specimen-Linked Database

The database 5 according to the invention is coupled to an Information Management System (IMS) 7. In one embodiment, the IMS 7 includes functions for searching and determining relationships between data structures in the database 5. In another embodiment, the IMS 7 displays information obtained in this process on an interface 6 of the user device 3. In one embodiment, the IMS 7 is stored within the server 4, and is accessible remotely by the user of the device 3 through the network 2. In another embodiment of the invention, the IMS 7 is accessible through a readable medium, which the user accesses through their particular device 3, such as a CD-ROM.

IMS 7's encompassed within the scope of the present invention include the Spotfire™ program, which is described in U.S. Pat. Number 6,014,661, the entirety of which is incorporated by reference herein. This database management software provides links to genomics data sources and those of key content and instrumentation providers, as well as providing computer program products for gene expression analysis. The software also provides the ability to communicate results and records electronically. Other programs can also be used, and are encompassed within the scope of the invention, and include, but are not limited to Microsoft Access, ORACLE and ILLUSTRA.

In one embodiment, the IMS 7 comprises a stored procedure or programming logic stored and maintained by the IMS 7. Stored procedures can be user-defined, for example, to implement particular search queries or organizing parameters. Examples of stored procedures and methods of implementing these are described in U.S. Pat. No. 6,112,199, the entirety of which is incorporated herein by reference.

In one embodiment of the invention, the IMS 7 includes a search function which provides a Natural Language Query (NLQ) function. In this embodiment, the NLQ accepts a search sentence or phrase in common everyday from a user (e.g., natural language inputted into an interface of a device 3) and parses the input sentence or phrase in an attempt to extract meaning from it. For example, a natural language search phrase used with the specimen-linked database 5, could be “provide medical history of patient at sublocation 1,1 of microarray 4591.” This sentence would processed by the search function of the IMS 7 to determine the information required by the user which is then retrieved from the specimen-linked database 5. In another embodiment of the invention, the search function of the IMS 7 recognizes Boolean operators and truncation symbols approximating values that the user is searching for.

In one embodiment, the search function of the IMS 7 generates search data from terms inputted into a field displayed on an interface 6 of a device 3 in the system 1 in a form recognized by at least one search engine (e.g., identifying search terms which are stored in fields in the database 5 or in the summary subdatabase), and transfers the search data to at least one search engine to initiate a search. However, in another embodiment, the search query is communicated through the selection of options displayed on the interface 6. For example, in one embodiment, search results are displayed on the interface 6, which may be in the form of a list of information sources retrieved by the at least one search engine. In another embodiment, the list comprises links which link the user to information provided by the information source. In a further embodiment, the search function of the IMS 7 removes redundancies from the list and/or ranks the information sources according to the degree of match between the information source and the search terms extracted, and the interface 6 displays the information sources in order of their rankings. Search systems which can be used are described in U.S. Pat. No. 6,078,914

In another embodiment, the search function of the IMS 7 searches a summary subdatabase of the database 5 to identify particular subdatabase(s) most relevant to the search terms which have been inputted by the user. In this embodiment, the search function of the IMS 7 restricts its search to subdatabases so-identified. In a further embodiment, the subdatabases searched by the IMS 7 can be defined by the user.

In one embodiment, relationships are defined by codes, such as SNOMED® codes, which can be inputted into the system by a user (e.g., on an interface of a user device). SNOMED® and SNOMED codes are described further in Altman, et al., Proceedings of American Medical Informatics Association Eighteenth Annual Symposium on Computer Applications in Medical Care. November 5-9, Washington D.C. pg. 179-183; Bale, Pathology.; 23(3): 263-267, 1991; Ball, et al., Computing pp. 40-46, 1999; Barrows, et al., Proceedings of American Medical Informatics Association Eighteenth Annual Symposium on Computer Applications in Medical Care, November 5-9, Washington D.C. pg. 21 1; Beckett, Pathologist, Vol. XXXI, No. 7, July 1977; Bell, Journal of the American Medical Informatics Association, 1(3): 207-217, 1994; Benoit, et al., Proceedings of the Annual Symposium of Computers Applications in Medical Care. 1992; pp. 787-788; Berman, et al., A SNOMED Analysis of Three Years' Accessioned Cases (40,124) of Surgical Pathology Department: Implications for Pathology-based Demographic Studies. Proceedings of American Medical Informatics Association Eighteenth Annual Symposium on Computer Applications in Medical Care. Nov. 5-9, 1994, Washington D.C. pg. 188-192; Berman, et al., Modern Pathology. 9(9): 944-950, 1996; Bidgood,. Meth. Inf. Med. 37: 404-414, 1998; Brigl, et al., International Journal of Bio-Medical Computing. 38: 101-108, 1995; Brigl, et al., Int J Biomed Comput. 37(3): 237-247, 1994;Campbell, et al., Methods Inf. Med. 37 (4-5): 426-39, 1998; and Campbell, et al., Proceedings of American Medical Informatics Association Eighteenth Annual Symposium on Computer Applications in Medical Care. November 5-9 1994, Washington, D.C. pg. 201-205, for example, the entireties of which are incorporated by reference herein.

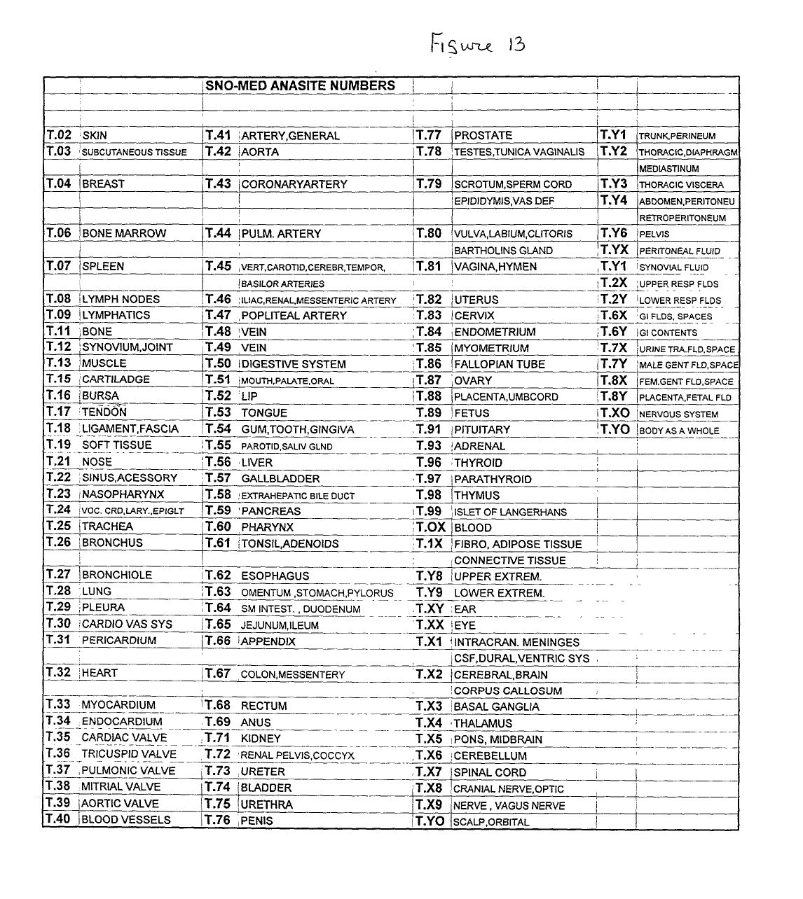

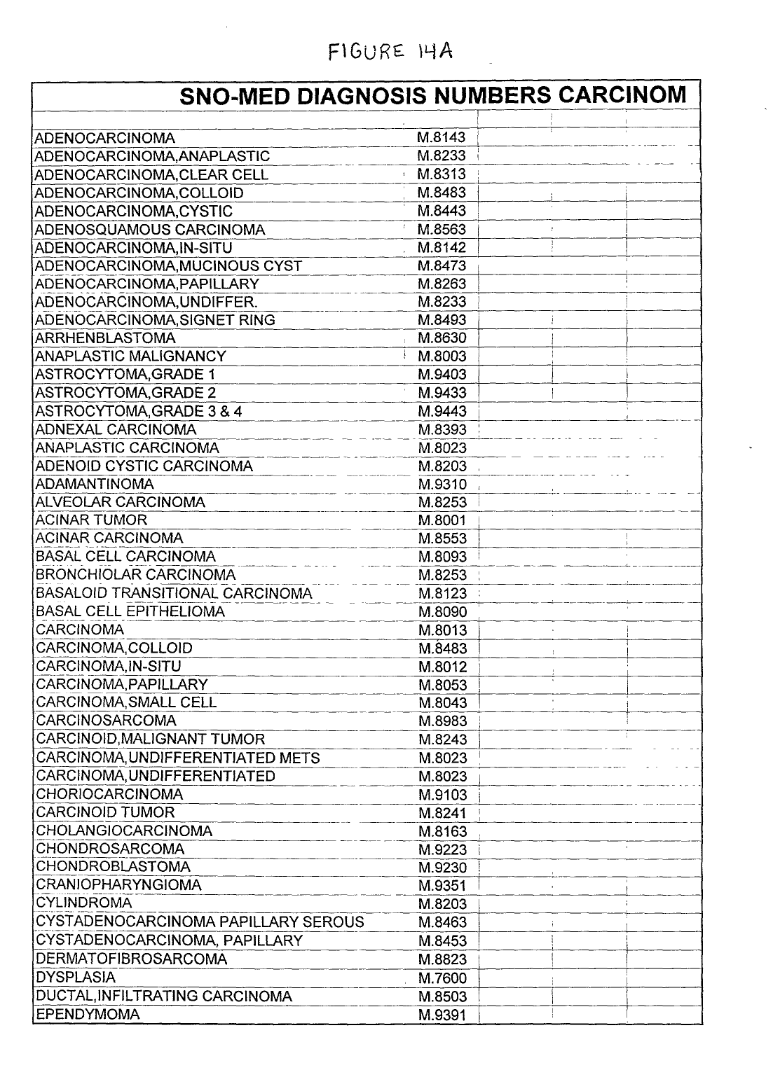

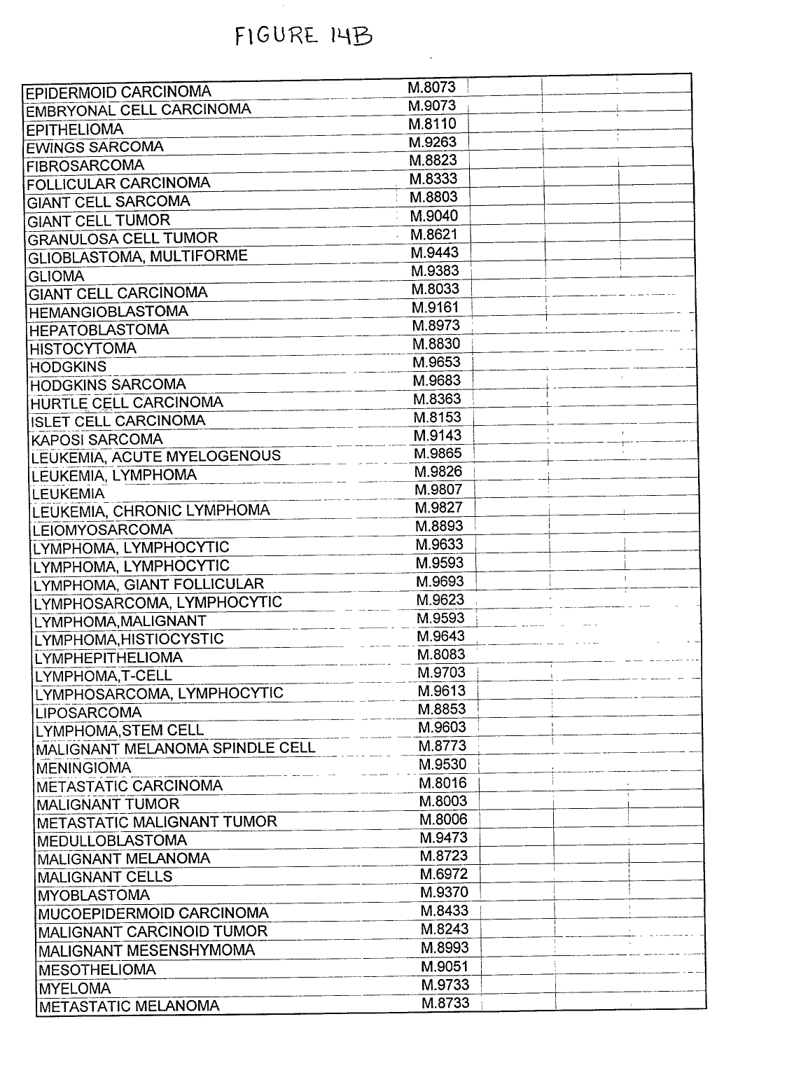

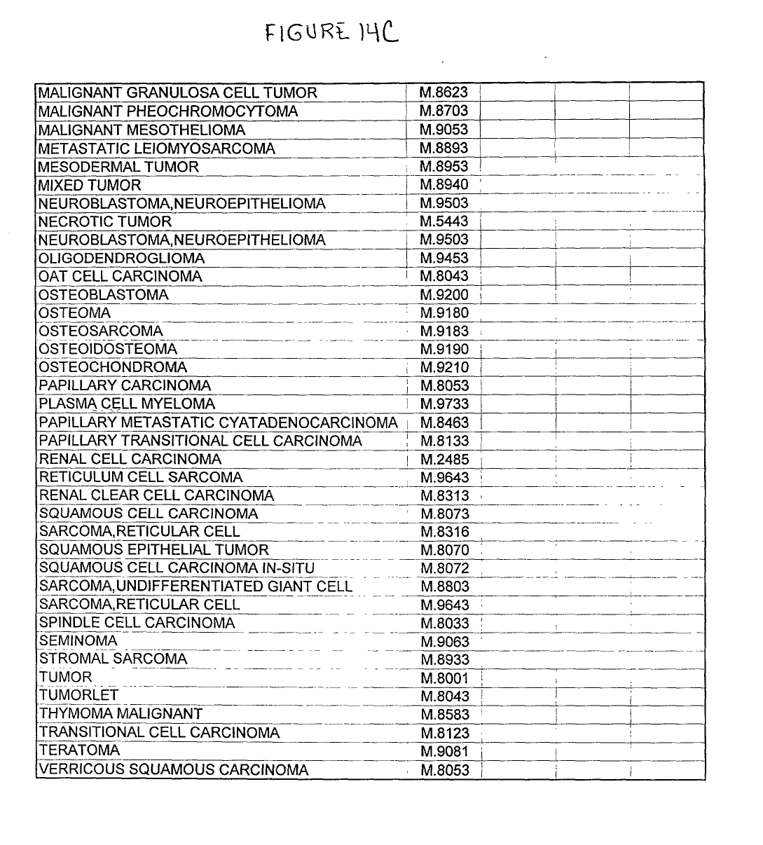

In a further embodiment of the invention, the IMS-7 includes a mapping function for mapping terms to particular tables within the database 5. Alternatively, or in addition to SNOMED®, other classification and mapping codes can be used (e.g., CPT, OPCS-4, ICD-9, and ICD- 10). In one embodiment, the IMS-7 comprises a program enabling it to read inputted codes and to access and display appropriate information from a relationship table. For example, in one embodiment, as shown in FIG. 13, unique SNOMED®) codes are assigned to tissues from specific anatomic sites, while in another embodiment, codes are assigned to tissues having specific pathologies (e.g., specific types of cancer) (see FIGS. 14A-C) and/or having selected pathologies (e.g., diagnostic codes are assigned to tissue samples/specimens which are the targets of specific types of cancer). In a further embodiment (not shown), tissue samples/specimens are cross-referenced using SNOMED® codes for both anatomic sites and diagnosis.

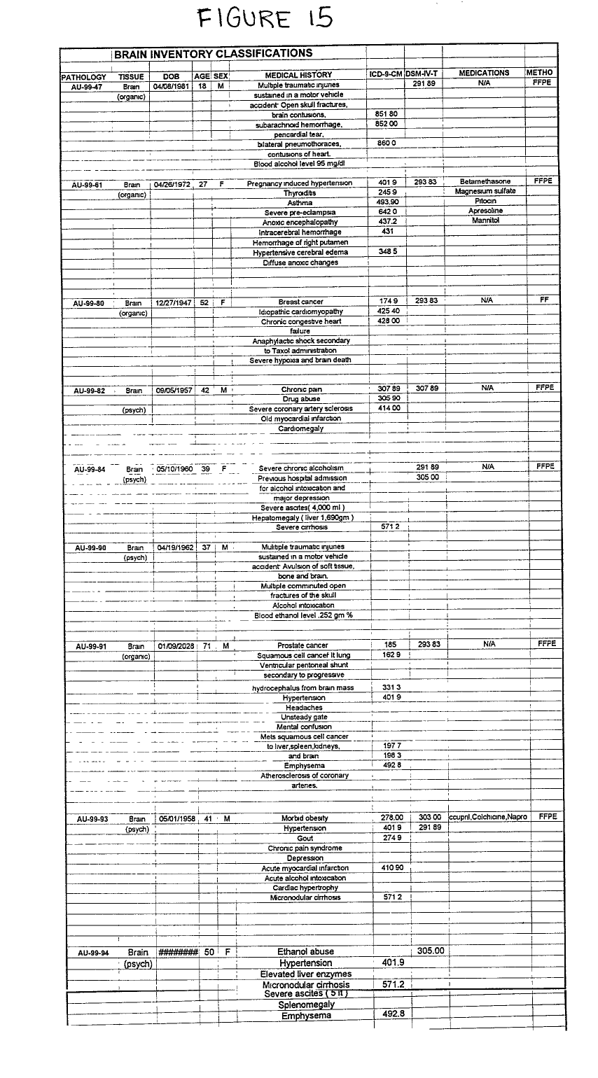

In a further embodiment, specimens/tissues are obtained from individuals having a neuropsychiatric disorder, and specimens/tissues on a microarray are cross-referenced in the database (i.e., linked to the database) according to the individuals' classification using DSM-IV-TR criteria. In another embodiment, specimens/tissues are linked to the database using ICD-9-CM criteria. In still another embodiment, as shown in FIG. 15, the specimens/tissues are cross-referenced using a number of criteria, such as tissue type, date of birth of the source individual, medical history of the source individual, ICD-9 criteria, DSM-IV TR criteria, Medications, and method of preparation. In a further embodiment, the ICD-9 and/or DSM-IV-TR criteria are indicated using codes. ICD-9 and DSM-IV TR codes are described at http://www.nzhis.govt.nz/projects/dsmiv-code-table.html, for example.

In addition to comprising a search function, the IMS 7 comprises a relationship determining function. In one embodiment, in response to a query and/or the user inputting information regarding a tissue into the tissue information system 1, the IMS 7 searches the database 5 and classifies tissue information within the database 5 by type or attribute (e.g., patient sex, age, disease, exposure to drug, tissue type, cancer grade, cause of death, and the like, and/or by codes, such as by SNOMEDT codes, ICD-9 codes, and/or DSM-IV-TR codes). In one embodiment, when all attributes have been defined and classified as characteristic of defined relationship(s), the IMS 7 assigns a relationship identification number to each attribute, or set of attributes, and signals representing these attribute(s) are stored in the database 5 (e.g., as part of the data dictionary subdatabase) where they are indexed by the relationship ID# and provided with a descriptor. For example, in one embodiment, the expression of a plurality of biological characteristics which have been classified as correlating to a disease state X (e.g., cancer) is assigned an ID# and a descriptor such as “diagnostic traits of disease X.”

In one embodiment, the relationship determining function of the IMS 7 employs a statistical program to identify groups of attributes as representing a particular relationship. In one embodiment, the statistical program is a non-hierarchical clustering program. In another embodiment, the clustering program employs k-means clustering.

The IMS 7 analyzes the relationships between data in the database 5 and/or new data being inputted, using any method standardly used in the art, including, but not limited to, regression, decision trees, neural networks, and fuzzy logic, and combinations thereof. In response to the results of this analysis, upon a query by a user, the system 1 displays at least one relationship or identifies that no discemable relationship can be found on the interface 6 of the user device 3. In one embodiment, the system 1 displays descriptors relating to plurality of relationships identified by the IMS 7 on the interface 6 as well as information relating to the statistical probability that a given relationship exists In one embodiment, the user selects among a plurality of relationships identified by the IMS 7 by interfacing with the interface 6 to determine those of interest (e.g., a relationship which is a disease might be of interest, while a relationship regarding hair color might not be). In another embodiment of the invention, rather than scanning an entire database 5, the IMS 7 samples the database 5 randomly until at least one statistically satisfactory relationship is identified, with the user setting parameters for what is “statistically satisfactory.” In a further embodiment of the invention, the user identifies particular subdatabases for the IMS 7 to search. In still another embodiment, the IMS 7 itself identifies particular subdatabases based on query terms the user of the system 1 has provided.

In one embodiment of the invention, the relationship of interest is used to provide a diagnosis of a disease (e.g., the relationship identified is a high correlation with a disease state). In another embodiment of the invention, the relationship of interest is used to identify the biological role of an uncharacterized gene, or to identify particular demographic factors (e.g., such as socioeconomic factors) associated a disease state.

In one embodiment of the invention, the IMS-7 system is used to identify populations of patients who share selected clinical characteristics by identifying sources of tissue samples who have these clinical characteristics. Clinical characteristics may be embodied in data which has already been entered into the database 5 or may be embodied in new data, which is being inputted into the system for validation. In one embodiment, populations of patients are identified who share a particular clinical history or outcome, a specific type of physiological response to a drug, either adverse or beneficial.

In another embodiment, the IMS-7 identifies relationships between sets of genes expressed or not expressed in tissues on one or more microarrays and clinical information relating to the patients from whom the tissues were obtained. For example, in one embodiment, the IMS-7 identifies relationships between a disease state (e.g., stroke) and genes expressed or not expressed during that disease state. For example, in one embodiment, the relationship determining function of the IMS-7 (for example, an application program which performs k-means clustering) is used to designate potential pathway genes, i.e., genes which are expressed during a disease and whose expression is related to the expression of other genes in the pathway.

Thus, in a very simple embodiment, where a stroke victim A expresses genes 1, 2, 3, 4, a stroke victim B expresses genes 1, 2, 4,7, 8, a stroke victim C expresses genes 1, 2, 4, 8, 9, 10, and normal patients D, E, and F express genes 2, 3, 8, the IMS-7 system would identify genes 1, 4, 7, 9, and 10 as potentially involved in a pathway of genes affected during stroke, and in certain embodiments, would rank genes 1 and 4 as being highly likely to be pathway genes. In a further embodiment, the IMS-7 system, in response to a user query would identify other patient parameters associated with the expression of genes 7, 9, and 10 and would perform clustering analyses to determine whether any relationships identified were statistically unlikely to arise by chance. For example, the IMS-7 system might identify that populations expressing genes 7, 9, and 10, in addition to stroke, suffer from cardiovascular disease.

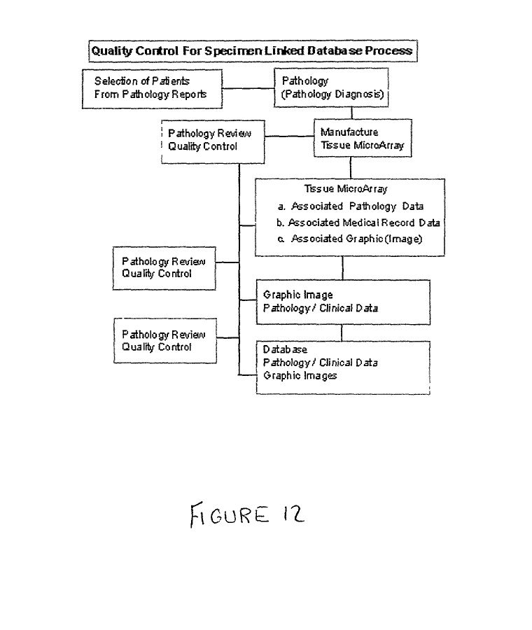

As illustrated by FIG. 11 A, in one embodiment, the user is able to view, print, permanently store, read, and/or further manipulate data displayed on the display 6 of his or her device 3. In this embodiment, the user is able to use the system 1 to investigate and define the relationships most relevant to tissues or diseases of interest (e.g., in the example shown in FIG. 11B, the relationship between medications being used and menstrual status, and further the relationship between menstrual status and other concurrent conditions, such as cardiac conditions experienced, hypertension, diabetes, pneumonia, etc.). In one embodiment, the user is also able to link to any database publicly accessible through the network 2, and to integrate information from such a database with the system 1's database 5 through the IMS 7. Thus, in one embodiment, information can be shared with other users and information from other users can be continuously added to the database 5.

One embodiment of the invention recognizes potential difficulties in enabling unrestricted access to the database 5, and encompasses providing restricted access to the database 5, and/or restricted ability to change the contents of the database 5 or records in the database 5 using the IMS 7 and/or a security application. Methods of providing restricted access to electronic data are known in the art, and are described, for example, in U.S. Pat. No. 5,910,987, the entirety of which is incorporated by reference herein.

### Organizing and Displaying Information on Graphical User Interfaces

The tissue microarrays 13 of the present invention can be used for diagnosis, prognosis, therapy, and research. The result of an analysis relating to any, or all of, the sublocations 13s on a microarray 13 can be compared and correlated with clinical, pathological, phenotypic, genomic, structural information, or any other information about the tissue stored within the specimen-linked database 5. Any number of microarrays 13 may be used, either in parallel or serially, in conjunction with the information provided by the database 5. Information from a single tissue sample may also be compared to pre-existing information on tissues in tissue microarrays 13 stored in the database 5.

In one embodiment, the system 1 allows the user to integrate and visually analyze in a single workspace, i.e., an interface 6 displayed on the display of the device 3, information contained in the tissue database 5 that is related to tissues of interest on a microarray 13 being analyzed by the user. In this embodiment, the IMS 7 further includes a linking application which links information in the database 5 to the interface 6 of a user device 3.

In one embodiment of the invention, the substrate of a tissue microarray 13 comprises coordinates or values for each sublocation 13s. Each coordinate can be related to information in the database 5 (e.g., a record or file). An identifying number 43i on the substrate can be used to identify the microarray 13 and information relating to the tissues on the microarray 13 (e.g., records or files within the database 5 can be indexed using the identifier 43i).

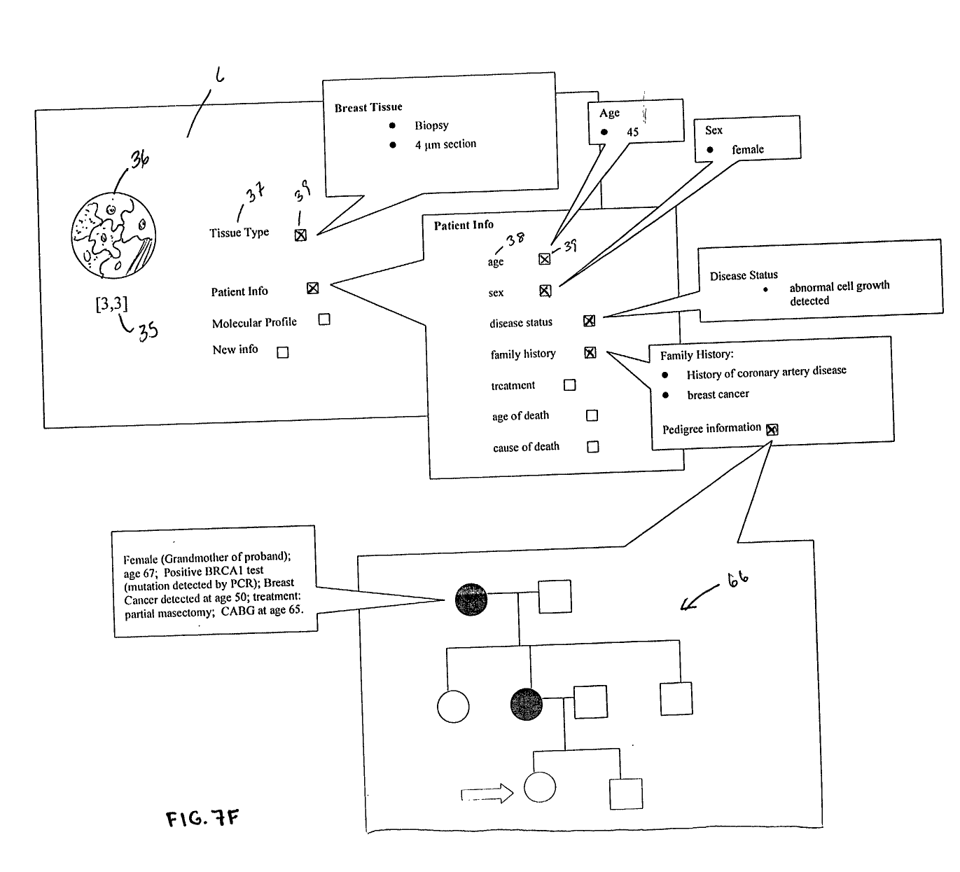

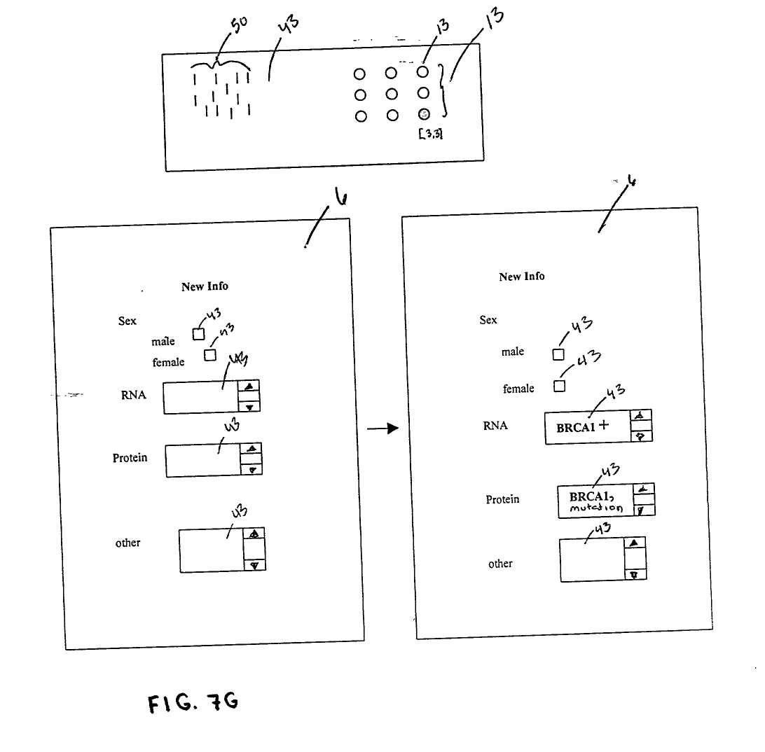

As shown in FIG. 6 and FIGS. 7A-7G, in one embodiment, a series of interfaces 6 for displaying information obtained from tissue microarrays 13 are provided to a user of the system 1 who has been provided with access to the database 5. Access to the interfaces 6 can be provided by providing the user with a locator, e.g., such as a URL, which can link the user directly to an overview interface (e.g., a homepage of a website) which summarizes the types of information contained within the database 5. However, in one embodiment, access to the database 5 itself and the IMS 7 requires the user to have access to the microarray identifier 43i (see, FIG. 6, STEP 1).

In one embodiment, the microarray identifier 43i is a string of alphanumeric characters uniquely identifying the microarray 13, while in another embodiment (shown in FIG. 8), information relating to the identity of the microarray 13 is encoded on a substrate 43 comprising the microarray 13 (e.g., encoded in a microchip or radiotransmittor, or in a bar code) and the information is automatically conveyed to the system 1 though a receiver 48 which receives the encoded information and which is in communication with the system 1. Access to the microarray identifier 43i therefore can be provided by providing the user with printed matter comprising a representation of the identifier 43i, by providing the identifier 43i verbally (e.g., by providing the user with a toll free phone number), or through an electronic means of communication, such as electronic mail. Alternatively, the identifier 43i can be provided by physically providing the user with the microarray 13 (i.e., where the identifier 43i is part of the substrate 43).

In one embodiment, accessing the overview interface 6 results in a field 35 being displayed for inputting the microarray identifier 43i (e.g., STEP 2 of FIG. 6, FIG. 7A). By inputting the identifier 43i into the field 35, the user accesses the database 5 comprising information relating to the particular microarray 13 identified by the identifier 43i (STEP 3 of FIG. 6 and also FIG. 7B).

In STEP 4 (FIG. 6, FIG. 7C), after the identifier 43i is inputted, another interface 6 is provided displaying coordinate links 35 corresponding to the coordinates of sublocations 13s on the particular microarray 13 which was identified by the identifier 43i. Each coordinate link 36 links the user to at least a portion of the database 5 comprising information relating to a particular sublocation 13s on the microarray 13. Coordinate links 35 according to the invention or a bold or otherwise distinctive font (e.g., different from the font of surrounding text), by underlining, by an icon, picture graphic (which may be a blinking graphic), or some other visual indication. Links 35 encompassed within the scope of the invention, include, but are not limited to, vertical links, circular links, horizontal hyperlinks, and combinations thereof Methods for providing links are known in the art and are described in, for example, U.S. Pat. No. 5,708,825, the entirety of which is incorporated by reference herein.

Coordinates links 35 can be displayed on the interface 6 in the form of a list, a table, or other arrangement. In one embodiment of the invention, coordinate links 35 are displayed as positional relationships as different sublocations 13s on the microarray 13. For example, coordinate links 35 can be displayed in rows and columns which pictorially represent the arrangement of sublocations 13s on the microarray 13. In one embodiment, each coordinate link 35 is in proximity to an image 36 of the tissue at the corresponding sublocation 13s of the microarray 13. For example, an image of a tissue at a sublocation 13s having the coordinates [3,3] is displayed on the interface 6 at coordinates [3,3] of the graphical image 39.

In one embodiment, the tissue image 36 is recorded by an optical system which has been, or is, in communication with the tissue microarray 13 (see, e.g., FIG. 8). In another embodiment, the tissue image 36 represents live optical data currently being collected by an optical system. In one embodiment, the image 36 of the tissue is itself associated with the link for accessing the database 5 (e.g., clicking on the tissue image will display an interface 6 presenting information related to that tissue), while in another embodiment, coordinate links 35 are displayed in proximity to the representation of the tissue (see, FIG. 7E).

It should be obvious to those of skill in the art that the exact arrangement of coordinate links 35 is not critical and can be modified, and that such modifications are encompassed within the scope of the invention. For, example, in one embodiment, the interface 6 comprises a field for entering coordinates on the tissue microarray 13 identified by the user (e.g., for example by using an microarray locator 45, such as the one shown in FIG. 2B). STEP 4 can therefore include providing a microarray locator 45 to overlay a tissue microarray 13 allowing the user to identify a coordinate of interest (e.g., the location, on an x, y coordinate system, of a sublocation 13s within a microarray 13 expressing biological characteristics of interest). In another embodiment, the tissue microarray 13 includes at least one orientation position (e.g., a tissue location stained or stainable with a “control reactive “molecule” (e.g., antibody, enzyme, dye, nucleic acid, and the like)) for orienting and manually determining coordinates on the tissue microarray 13, and STEP 4 includes the step(s) of identifying the orientation positions on the microarray 13. In still further embodiments, a substrate 43 comprising a microarray 13 being analyzed comprises encoded addressing information which is received by a receiver 48 in communication with the system 1 (see, FIG. 8, for example).

In STEP 5, at least one coordinate link 35 is selected (FIG. 7D), and in STEP 6, in response to the user selecting particular coordinate link(s) 35, the system 1 displays information relating to the tissue at the sublocation 13s identified by the coordinate link 35 (FIG. 6, FIG. 7E). In one embodiment, the displaying step further comprises the step of displaying information category options 37 (see FIG. 7E-7F). Information category options 37 are links to specific portions of the database 5 comprising the information categories. In one embodiment, shown in FIG. 7E, information category options 37 include a tissue type option, a patient information option, molecular profile option, and new information option (“new info”). Information category options 37 can further include information category suboptions 38, further defining specific portions of the database 5 which the user seeks access to.

In STEP 7, at least one information category 37 is selected (for example, by checking option boxes 39 provided in proximity to the information categories 37), causing the system 1 to display other information interface(s) 6 displaying information relating to the particular information categor(ies) selected (STEP 8; see also callouts in FIG. 7F, each callout represents interfaces 6 displayed upon selection of the indicated information categories 37). In one embodiment, as part of the displaying process, additional information subcategories 38 can be displayed which can be further selected (STEPS 9 and 9A; see also FIG. 7F).

In a further embodiment of the invention, a subcategory option 38 is provided which comprises provides a link to pedigree information. Selecting this subcategory option 38 causes the system 1 to display an interface 6 providing a pedigree chart 66, e.g., with boxes and circles representing individual family members and lines connecting the boxes and circles representing relationships between family members. In one embodiment, clicking on a box or circle will link the user to another interface 6 on which detailed information relating to the individual family member is displayed, and/or which provides more links representing options which the user can select to display molecular profiling information or patient information relating to the individual family member. The arrow on the pedigree chart represents the proband, e.g., the source of the tissue sample at coordinate [3,3] of the microarray 13.

In a further embodiment, the selection STEP 7 includes selecting the information category option 38, “new info.” Selecting the new info category option 37 displays at least one interface 6 on which the user can add new information (e.g., in fields 43) to be included in the database 5 (STEPS 9B-9C; see also FIG. 7G). In one embodiment, the new information is molecular information relating to the expression of nucleic acids, proteins, and other biomolecules in the tissue microarray 13 or in a tissue sample, or other sample (e.g., a nucleic acid sample or protein sample) being compared to the tissue microarray 13.

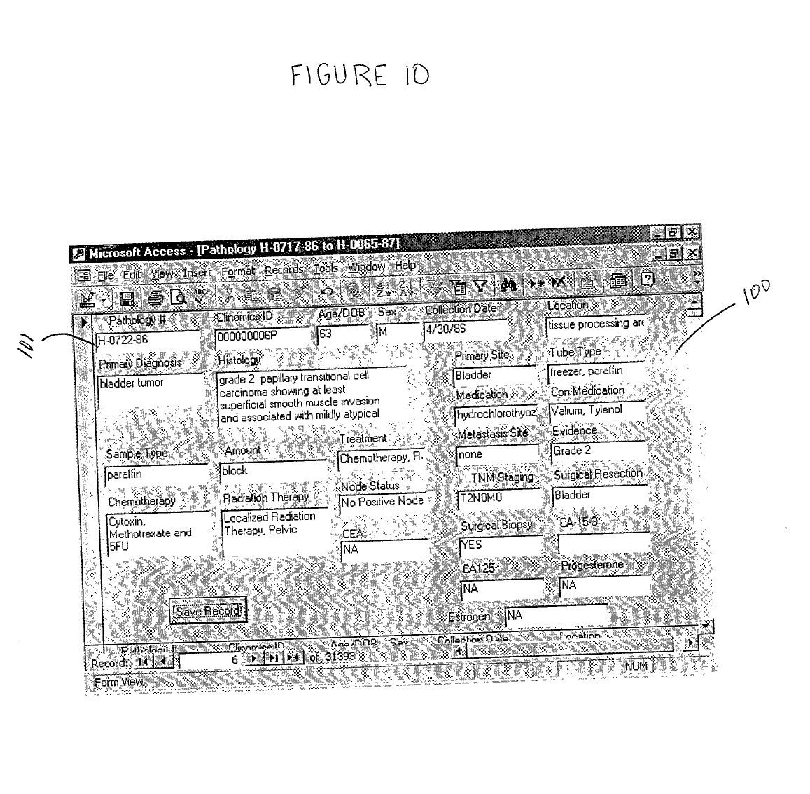

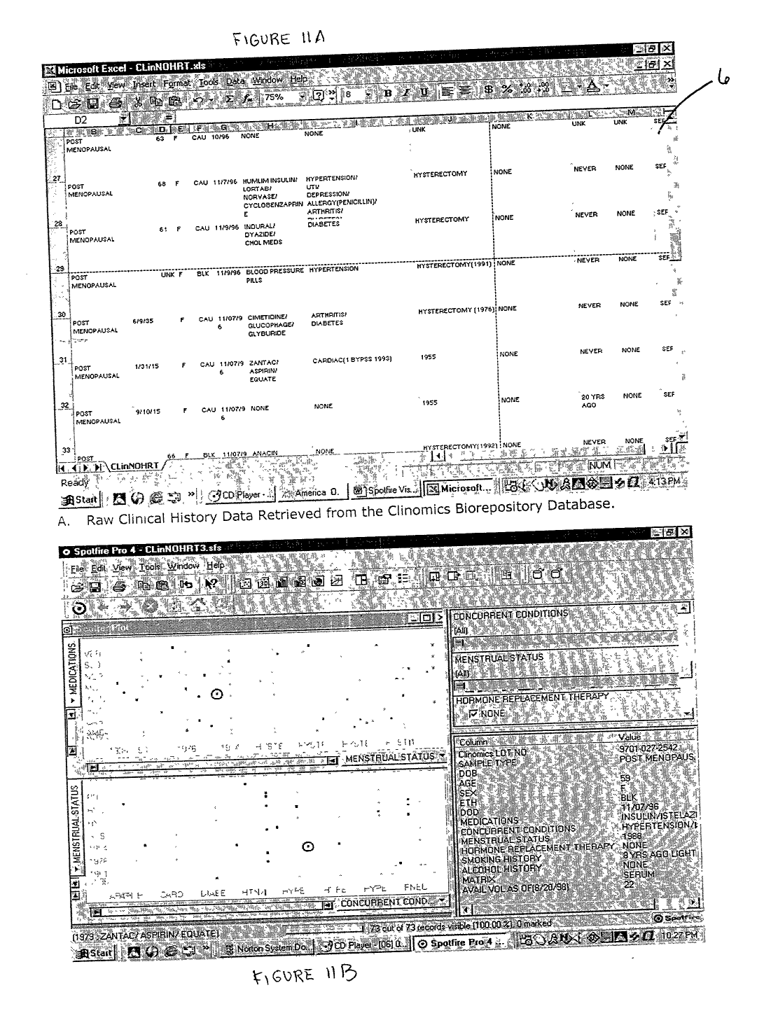

As shown in FIG. 7G, in one embodiment, both a nucleic acid microarray 50 and a tissue microarray 13 are provided on the same substrate 43, and information relating to the expression of a disease-related biomolecule is determined (e.g., in the embodiment shown in FIG. 7G, the disease-related biomolecule is the product of the BRCA1 gene). The user inputs information relating to the expression of these biomolecules into new information fields 43 and this information is in turn communicated to the IMS 7 and can be stored in the database 5. In one embodiment, the information is stored in a temporary portion of the database 5 until validated (e.g., by repeating the analysis with another tissue microarray from the same recipient block). provides access to a particular specimen-linked database 5. For example, as shown in FIG. 10, in one embodiment, an interface 100 is provided which allows a user to access a genomic medicine database as described above. In this embodiment, the interface 100 is displayed in response to a user entering an identifier corresponding to a microarray 13 being evaluated. In response, the system displays on the display of the user's user device an interface which comprises a number of fields 101 displaying information relating to one or more sublocations on the microarray 13. For example, as shown in FIG. 10, in one embodiment, fields include a pathology field (for example, displaying a SNOWMED code corresponding to a particular pathology), a primary diagnosis field (e.g., bladder tumor), a description of the sample type field (e.g., paraffin, in this example), a histology field, treatment regimen fields (e.g., chemotherapy, radiation therapy), node status, expression of particular cancer antigens (e.g., CEA expression), the primary site of pathology (e.g., bladder), medications being taken, any sites of secondary metastases, TNM staging, how the sample was obtained (e.g., through a surgical biopsy), grade, concurrent medications (i.e., medications not being taken which are not directed to the treatment of a bladder tumor, such as valium, and tylenol), and the like, for an individual sublocation on a microarray. This information can be used to correlate the expression of a marker (for example, p53 expression, simultaneously with patient information, medical information, pathology information, and other genomic information relating to the source of tissue at the particular sublocation on the microarray.

### Molecular Profiling Using the Tissue Information System

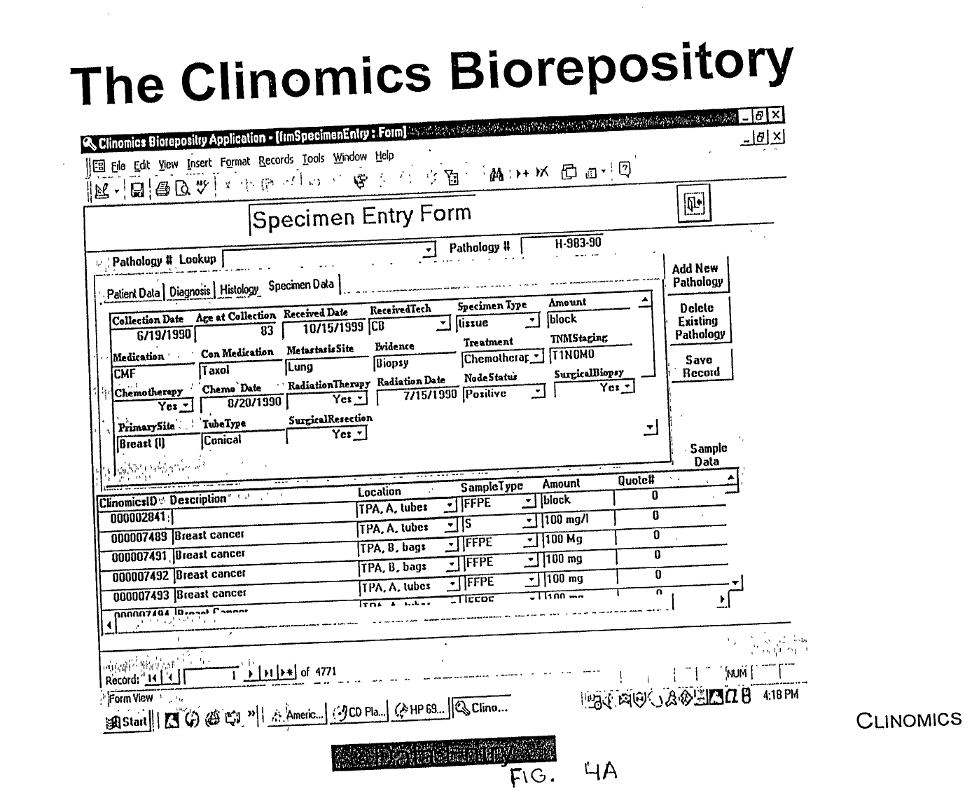

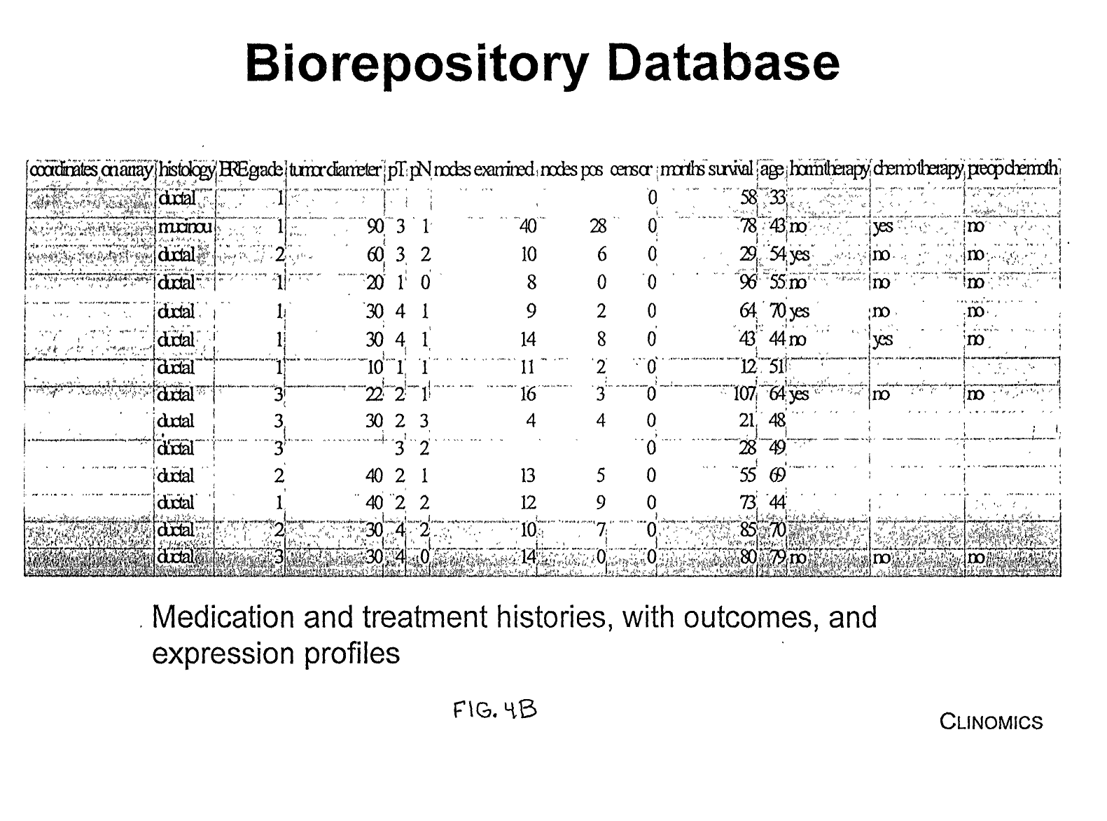

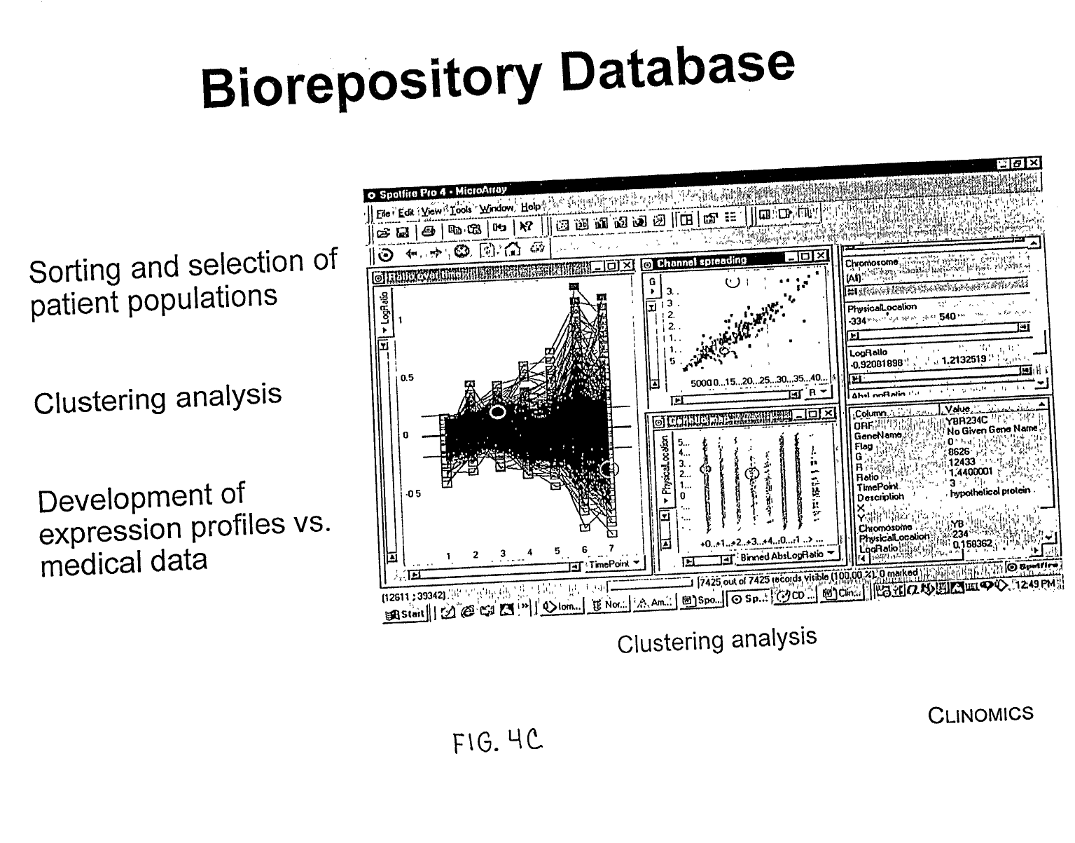

New information can be used to generate or refine molecular profiles. Such molecular profiles can be displayed on yet another interface 6 (see, for example, FIG. 4C). In one embodiment of the invention, a plurality of microarrays are assayed, serially, or in parallel, and the results from this analysis are evaluated by using the relationship determining function of the IMS 7.

In one embodiment, different types of microarrays are screened to provide molecular profiling data, including any of: a tissue microarray 13, a cell line microarray, a nucleic acid microarray (e.g., a genomic microarray, a cDNA microarray, an oligonucleotide microarray, an aptamer microarray), a peptide microarray, or other small biomolecule array. In another embodiment, a tissue microarray 13 is screened in parallel with a nucleic acid microarray comprising ESTs (expressed sequence tag sequences) to identify ESTs which hybridize to nucleic acid samples from an individual having a particular disease (or other biological characteristic of interest) and to validate that an EST so identified is expressed in a statistically significant proportion of tissue samples in microarrays 13 to be diagnostic (e.g., in a population set provided to the user or in a cumulated set representing analyses performed by multiple users. Similarly nucleic acid arrays comprising SNPs can be analyzed in the same way. In one embodiment, SNP data is entered into the database 5 and communicated to the IMS 7 which correlates allelic frequency of a particular SNP with patient information (e.g., particular disease states, ethnic background).

In one embodiment, the IMS 7 implements a statistical program to identify relationships between biological characteristics of tissues on the microarray, including information from molecular profiling analyses. In this embodiment, the IMS 7 using an application for implementing a nonhierarchical statistical analysis of data, such as k-means clustering. In another embodiment, the IMS 7 determines the frequency at which particular biological characteristics are expressed, and correlates frequency information to any of: disease diagnosis, progression, recurrence, response to treatment, and the like

### Identifying and Validating Diagnostic Molecules Using the Tissue Information System

In one embodiment, the system 1 provides a way to identify and validate diagnostic molecular. For example, in a first phase of this embodiment, test probes specifically reacting with a gene or gene product are used to evaluate microarrays (tissue microarrays, cell line microarrays, nucleic acid microarrays, peptide microarrays, and/or other small biomolecule arrays) and to identify a biomolecule or set of biomolecules whose expression is diagnostic of a trait (e.g., by determining which molecules on the microarray are always present in a disease sample and always absent in a healthy sample, or always absent in a disease sample and always present in a healthy sample, or always present in a certain form in a disease sample and always present in a certain other form in a healthy sample, (or where there is a statistically significant difference in the expression or form of such molecules in these samples as determined by routine statistical testing to within 95% confidence levels)).

In the second phase of this embodiment, test probes identifying diagnostic biomolecules are contacted to tissue microarrays according to the invention, to identify the presence and/or form, and/or location of the diagnostic biomolecules in microarray(s) comprising different types of healthy or diseased tissues (or at least including sublocations comprising tissue from which the disease and patient samples were obtained for testing in phase one). In this way, the correlation between the expression of the diagnostic biomolecule(s) identified and the disease state is validated. In one embodiment, data from both phase one and phase two are inputted into the database 5 and the IMS 7 are used to determine the relationship(s) between the data obtained in phase one and phase two (e.g., whether the data obtained is diagnostic), and the data validating the diagnostic biomolecule is inputted into the database.

In another embodiment of the invention, the role of diagnostic molecule(s) are evaluated by comparing the expression of the molecule(s) in different sublocations on the microarray(s) with information in a database 5 relating to the type of tissue, its developmental stage, or to other traits of the individual(s) from which the tissue is obtained.

In a further embodiment of the invention, the expression of the diagnostic molecule is examined in a microarray comprising tissues from a drug-treated patient and tissues from an untreated diseased patient and/or from a healthy patient, and the efficacy of the drug is monitored by determining whether the expression profile of the diagnostic(s) molecule returns to that of a healthy patient. In one embodiment of the invention, a test tissue is obtained from a patient treated with a drug and a microarray is provided comprising at least both disease tissue and healthy tissue of the same type as the test tissue. In this embodiment, the expression of the diagnostic molecule(s) in the test tissue is compared with the expression pattern in the disease or healthy tissue using the system 1, and a drug is identified as useful for further testing when the expression pattern in the test tissue is substantially the same as the expression pattern within the healthy tissue, as determined using the system 1. In another embodiment, information validating a drug, and including testing data, is stored within the database 5.

### Diagnostic Matrix For Classifying Biological Characteristics

In one embodiment, a panel or collection of tissues samples is obtained representing a plurality of different stages of a disease (e.g., such as cancer) which is used to generate the sublocations of an disease tissue microarray 13 (e.g., an oncology tissue micrarray 13). In order to establish a panel which is useful for predicting the prognosis of a given cell or tissue sample, a scoring method or information matrix is established which relates the expression of a first biological characteristic (e.g., level of expression cancer-specific marker, as reflected by antibody staining) to a second biological characteristic (e.g., localization of the cancer-specific marker). In one embodiment, data relating to the information matrix is stored in the database 5 of the system 1.

For example, in one embodiment, the biological characteristic is nuclear staining for a polypeptide, and the tissue panel is classified according to the percentage of cells expressing the polypeptide and how intensely those cells express the polypeptide. Cancer cells are placed into groups based on 1) a range of percentages of cells expressing the marker polypeptide, for example, 5 groups of <20%, 20% to <40%, 40% to <60%, 60% to <80%, and 80% to 100%, and 2) a range of degrees of staining intensity, for example, 4 groups ranging from light staining, light to medium staining, medium to dark staining and dark staining.

These quantities are used to place the biological characteristic for a given test sample into one of a number of categories that considers both elements of the characteristic being classified. The number of categories in this case is determined as the product of the number ranges of percentages and the number of ranges of staining intensity (in the present example, there would be 20 categories; a single further category can be added that includes cancer cells with no nuclear staining for the polypeptide). The categories are illustrated below in Table 1. In reference to the table, for example, a sample with 35% of cells staining light to medium would be scored 2/2. One should also note that within a given tissue sample there are most frequently more than one cell type. The scoring of cells in the tissue samples can be done individually in those cases in which the tumor retains morphologically distinct cell types. Thus, for a given tissue sample, one may have separate expression characteristic scores for, e.g., epithelial cells, glandular cells and inflammatory cells; or other indicia of morphology that reflect any of the grading systems for abnormal cell growth described above (e.g., TNM, Duke's stage, Gleason stage, BRE stage, and the like). By correlating the matrix data (e.g., as in the Table below) with the grade of cancer, a user of the microarray 13 can stage a test tissue by identifying the two biological characteristics expressed in the tissue.

TABLE 1
Percent (%) of Cells Staining

| Degree of | | | | | |
|---|---|---|---|---|---|
| Staining | <20% | 20%-<40% | 40%-60% | 60%-<80% | 80%-100% |
| Light | 1/1 | 1/2 | 1/3 | 1/4 | 1/5 |
| Light/ | 2/1 | 2/2 | 2/3 | 2/4 | 2/5 |
| Medium | | | | | |
| Medium/ | 3/1 | 3/2 | 3/3 | 3/4 | 3/5 |
| Dark | | | | | |
| Dark | 4/1 | 4/2 | 4/3 | 4/4 | 4/5 |

Thus, when the score assigned to a patient's tissue sample for a given biological characteristic (e.g., a cancer specific marker) substantially matches the score of a test sample for the same biological characteristic (i.e., is not statistically different based on routine statistical tests to within 95% confidence levels), the prognosis of the patient's disease is correlated to that of the patient from whom the standard sample was obtained. The accuracy of prognosis value of increases as more markers are considered. In the methods of the invention, the ability to screen serial sections of a tissue microarray 13 with multiple probes, and to correlate the expression characteristics of those probes on a one microarray 13 with the same probes on another microarray 13 or a plurality of other microarrays 13, facilitates the generation of a molecular profile representing multiple biological characteristics which is useful in diagnosis, prognosis, guidance of treatment and prediction of a patient's relapse.

In one embodiment, information relating to a diagnostic matrix established for a given type of cancer and a given microarray 13 is stored in the database 5, along with all other information available relating to the patient from which a particular tissue sample came. However, in addition to the information regarding each tissue sample, the database 5 can contain information on other tissue samples not included on the particular microarray(s) 13 examined by a given health care worker. These data provide depth to the database 5 beyond the samples on a given microarray 13, and enhances the statistical reliability of decisions based upon a given microarray 13.

For example, a collection of 250,000 or more samples of breast cancer tissue may be available. A given tissue microarray 13 will not necessarily have samples of all of them, but will more likely have a subset of those tissue samples. Therefore, there can be multiple microarrays 13, each comprising a different subset of the total collection of samples. As each subset microarray 13 is analyzed for different markers, the data are reported back to the database 5. When a clinician reports data back to the database 5 for a given marker, he or she can be informed of whether other clinicians have examined the same marker in other samples on other subset microarrays 13, by querying for this information using the IMS 7.

The information for those subset microarrays 13 examined for the same marker can then be provided to clinicians for use in diagnosis or prognosis of their patient's condition. The result of this is that examination of an microarray 13 of, for example, 500 tissue samples can effectively yield information on many more tissue samples in other subset microarrays 13. The predictive value of a standard panel and the database 5 associated with it increases as data is reported back to the database 5 for individual markers.

In one embodiment of the invention, the information matrix is displayed as a grid, however, in another embodiment of the invention, the information matrix is accessed, when the user inputs information relating to a biological characteristic obtained into field(s) on the interface 6 of a user device 3, and a linking application communicates this information to the IMS 7, which displays a diagnosis/prognosis based on the inputted information.

### Automated Molecular Profiling System

In one embodiment of the invention, collection of molecular profiling data is at least partially automated (as shown in FIG. 8). In this embodiment, a tissue microarray is provided in communication with an optical system. The optical system comprises a light source 67 in communication with at least one light directing element 68 for directing light to a substrate 43 comprising the tissue microarray 13 (e.g., a glass slide) and at least one light directing element 68 for directing light from the tissue microarray 13 to a detector 69. In one embodiment, the detector 69 detects scanned light from at least one sublocation 13s at a time (e.g., emitted light, reflected light and/or scattered light), and converts this light into a signal using a processor 47 in communication with the detector 69. The signal is converted into optical information relating to all, or selected wavelengths of light, transmitted by the tissue. In one embodiment the optical information is an image of the tissue, while in another embodiment, the optical information is spectral information.

In one embodiment, the detector 69 detects light from a reactive molecule used to label any of protein, nucleic acids, and other biomolecules, and the optical expression data from at least one sublocation 13s is displayed on an interface 6 of a device 3 connected to the network 2.

In one embodiment, optical expression data is superimposed on a representation of the tissue microarray. Expression data can be automatically or manually inputted into a new information subdatabase of the database 5 (e.g., a temporary database), and can also, or alternatively, be saved in a molecular profiling subdatabase.

In a further embodiment of the invention, the substrate comprising the microarray 13 comprises an identifying element 43i (e.g., a microchip, electronic transducer element, or radio frequency transmitter) and transmission of an identifying signal (e.g., an electromagnetic signal or a radio signal) identifying the particular tissue microarray being examined is communicated to the processor 47. In one embodiment of the invention, the processor 47 is connected to the tissue information system 1 (e.g., through the network 2) and the system 1, upon receiving the identifying signal displays an interface 6 comprising a plurality of coordinates, each coordinate providing a link to the database 5 comprising information about tissue at the coordinate (i.e., as shown in FIGS. 7i-7G).

### System For Ordering Customized Tissue Microarrays

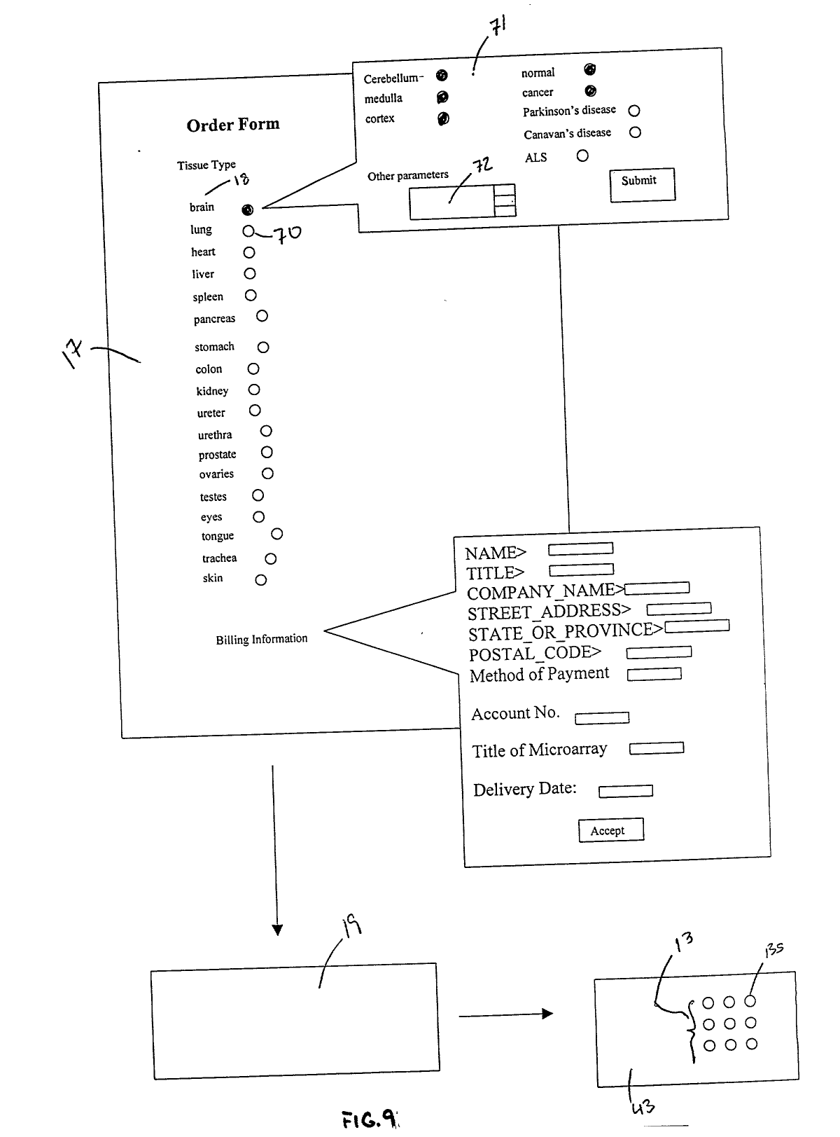

The invention further provides a system for ordering customized microarrays 13 electronically. In one embodiment, as shown in FIG. 9, a first user is provided access to an interface 17 which displays identifiers 18, each of which identifies a different tissue type. The first user identifies tissue types of interest (e.g., by checking any of a plurality of circles 70 provided alongside an identifier 18 which identifies the tissue type), or obtains more information about the tissue types (e.g., in this embodiment, the tissue type identifier 18 is itself a link which, when selected, causes the system to display another interface (not shown) providing information about the tissue type/source, such as patient data, molecular profile data, and the like).

In one embodiment, the interface 17 further provides an option to select tissue type(s) as well as the option to select more links, or to continue searching to identify other tissues of interest (not shown). Selection of tissue type(s) is communicated to a microarray generator 19 which constructs the tissue microarray 13.

In one embodiment, the interface 17 accessed by the first user provides field(s) 72 to enter query terms, and the system 16, displays tissue information relating to these query terms. For example, in one embodiment, the user enters keywords requesting information relating to lung cancer and exposure to asbestos, and the system displays identifiers 18 identifying tissues obtained from patients with lung cancer who have been exposed to asbestos. Selection of any of the identifiers 18 will communicate a request to the microarray generator 19 to provide these tissue(s) on the microarray 13. Microarray generators 19 encompassed within the scope of the invention include, but are not limited to a second user, a microarray generating system (e.g., such as a robotic tissue arrayer), or a combination thereof.

In one embodiment, the microarray generating system is a robotic system which selects donor blocks and generates recipient blocks based on commands of the first user which have been communicated to the generator 19. Methods of programming robotic systems to perform designated tasks are described, for example, in U.S. Pat. No. 4,835,730, the entirety of which is incorporated by reference herein. In one embodiment, the database 5 additionally includes an “assembly sequence” subdatabase, which includes information relating to the tasks to be performed by the robotic system, as well as subdatabases comprising information relating to the assembly locations of the donor and recipient block(s), and other parts of the automatic tissue microarrayer. In this embodiment, the server 4 additionally comprises software routines which control how these tasks are performed.

In another embodiment, the interface 17 further requests information from the first user such as billing information (credit card, account number, and the like), address, date required, and other shipping information. In further embodiments, the user is also provided with the option to select nucleic acid arrays, peptide arrays, and/or other small biomolecule arrays, which may be arrayed on the same or different substrates as the tissue microarray 13.

### Kits

The invention further provides kits. A kit according to the invention, minimally contains a tissue microarray 13 and provides access to an information database (e.g., in the form of a URL and an identifier which identifies the particular microarray being used, and/or a password). In one embodiment, the kit comprises instructions for accessing the database 5, or one or more molecular probes, for obtaining molecular profiling data using the microarray 13, and/or other reagents necessary for performing molecular profiling (e.g., labels, suitable buffers, and the like). In one embodiment of the invention, the components of the kits are customized by a second user receiving information from a first user as described above.

### Reports

The invention also encompasses production of reports or summaries of the information relating to tissue microarrays 13 of the invention which have been organized using system 1. In one embodiment, a screen to determine the expression of biological characteristics of tissues on the microarray 13 and/or test tissues is performed, and results of that screen are reported (e.g., in printed or electronic, verbal form).

More generally, the report may include information describing the common properties of the tissues in the microarray 13, and/or an analysis of differences between the tissues. In one embodiment, the report or analysis is communicated to a first user of the microarray 13 after the first user communicates to the system 1 (and/or a second user), the form in which the first user wishes the report (e.g., selecting particular biological characteristics the first user wishes reported on an interface displayed by the system 1).

What is claimed is:

1. A tissue information system, comprising:
a specimen-linked database comprising information about at least one tissue microarray identified by an identifier; and
at least one user device connectable to the network, for displaying an interface onto which a user can input said identifier, said inputting enabling said user to access the database.

2. A method of obtaining tissue information, comprising:
providing a user with a tissue microarray;
providing the user with an identifier which identifies the microarray;
providing the user with access to the system of claim 1, and displaying the interface; and
allowing the user to input the identifier into the interface displayed by the system wherein the system, in response to the user inputting said identifier, displays tissue information relating to the tissue microarray identified by the identifier.

3. A tissue information system, comprising a database, the database comprising a diagnostic matrix which relates the expression of a biological characteristic of a tissue to a disease state; and
a user device connectable to the network, said user device for displaying an interface which enables the user to input information relating to the biological characteristic, in response to which inputting, the system displays information correlating the expression of the biological characteristic to a disease state.

4. A method for obtaining data about a sample in a microarray, said array comprising a plurality of tissue samples, the method comprising the steps of:
a) providing an interface on a display of a user device connectable to the network;
b) displaying a plurality of selectable coordinates on said interface, each coordinate representative one of the samples in the microarray and associated with a link for accessing a database, said database comprising information relating to said one of said samples in the microarray; and
c) allowing a user to select a link associated with one of said coordinates, thereby accessing said database and obtaining information about said sample.

5. A system for ordering customized tissue microarrays, comprising:
a database comprising information about a plurality of tissue samples; and
at least one user device connectable to the network for displaying an interface which provides a plurality of tissue links, wherein selecting one of the links enables a user to access information within the database relating to a tissue identified by said link, and to optionally request that said sample be provided on a tissue microarray.
